(19) United States
(12) Patent Application Publication
(10) Pub. No.: US 2026/0365676 A1
(43) Pub. Date: Nov. 19, 2026

(54) MACROCYCLE OXIDATIVE STRESS INDUCERS AND METHOD OF USE OF THE SAME

(71) Applicant: Georgia Tech Research Corporation, Atlanta, GA (US)

(72) Inventors: Adegboyega Oyelere, Atlanta, GA (US); Dipak Walunj, Atlanta, GA (US); Uche Arunsi, Atlanta, GA (US); Jeremiah Olugbami, Atlanta, GA (US)

(21) Appl. No.: 19/565,409

(22) Filed: Mar. 12, 2026

Related U.S. Application Data
(60) Provisional application No. 63/770,533, filed on Mar. 12, 2025.

(51) Int. Cl. C07F 1/12; A61K 47/52; A61P 33/06; A61P 35/00; C07D 407/14; C07D 413/14; C07D 493/08

(52) U.S. Cl. CPC C07F 1/12; A61K 47/52; A61P 33/06; A61P 35/00; C07D 407/14; C07D 413/14; C07D 493/08

(57) ABSTRACT

An exemplary embodiment of the present disclosure provides a compound of formula (I) having the following structure:

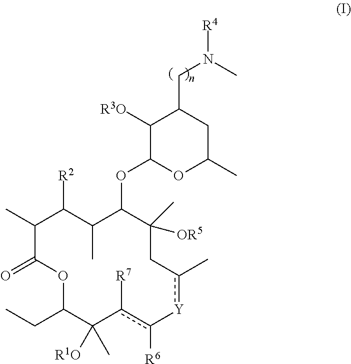

or a stereoisomer, pharmaceutically acceptable salt, oxide, or solvate thereof, wherein R¹, R², R³, R⁴, R⁵, R⁶, R⁷, Y, and [symbol] are as described herein. The present disclosure also provides compositions comprising these compounds and methods of using these compounds to up-regulate reactive oxygen species (ROS), particularly for the treatment of cancer or malaria.

### CROSS-REFERENCE TO RELATED APPLICATIONS

This application claims the benefit of U.S. Provisional Application Ser. No. 63/770,533, filed on Mar. 12, 2025, which is incorporated herein by reference in its entirety as if fully set forth below.

### FIELD OF THE DISCLOSURE

The various embodiments of the present disclosure relate generally to a composition of matter comprising macrocycle oxidative stress inducers and their use as anticancer agents that function by up-regulating reactive oxygen species (ROS) and ferroptosis in cancer cells and selectively accumulate within the tumors of the liver, lungs, and breast. The compounds inhibit the viability and proliferation of several cancer cells including prostate, lung, liver, and breast cancers.

### BACKGROUND

Dysfunction in the regulation of oxidation stress of cells, including cancer cells, can regulate cell proliferation and programmed cell death (PCD). Intracellular levels of reactive oxygen species (ROS) are key indicators of oxidation stress. ROS can play dual roles in cancer cell viability depending on their levels, type, source, and duration of exposure (Luo et al., “Self-Assembled Redox Dual-Responsive Prodrug-Nanosystem Formed by Single Thioether-Bridged Paclitaxel-Fatty Acid Conjugate for Cancer Chemotherapy,” Nano Lett. 16 (9): 5401-5408 (2016); Cai et al., “The involvement of ROS-regulated programmed cell death in hepatocellular carcinoma,” Crit Rev Oncol Hematol. 197:104361 (2024)). Below a certain threshold, ROS could promote the proliferation of cancer cells while above the threshold, ROS could trigger PCD in cancer cells (Nogueira and Hay, “Molecular pathways: reactive oxygen species homeostasis in cancer cells and implications for cancer therapy,” Clin Cancer Res. 19 (16): 4309-14 (2013); Hayes et al., “Oxidative Stress in Cancer,” Cancer Cell 38 (2): 167-197 (2020); An et al., “Oxidative cell death in cancer: mechanisms and therapeutic opportunities,” Cell Death Dis. 15 (8): 556 (2024)). Excessive levels of ROS could trigger various forms of PCD including apoptosis, ferroptosis, autophagy and necrosis (Porporato et al., “Mitochondrial metabolism and cancer,” Cell Res. 28 (3): 265-280 (2018). Several anticancer drugs trigger ROS generation as a direct or indirect mechanism of their cell cytotoxicity (Jiang et al., “Drug-induced oxidative stress in cancer treatments: Angel or devil?” Redox Biol. 63:102754 (2023); Yang et al., “The role of cellular reactive oxygen species in cancer chemotherapy,” J Exp Clin Cancer Res. 37(1):266 (2018)). Other drugs that work by triggering ROS generation are being investigated as potential anticancer agents. Endoperoxide containing drugs such as the antimalarial drug artemisinin and metallodrugs such as auranofin are examples of ROS triggering agents that have demonstrated potential as anticancer agents (Chen et al., “Artemisinin compounds sensitize cancer cells to ferroptosis by regulating iron homeostasis,” Cell Death Differ. 27(1):242-254 (2020); Xu et al., “Artemisinins as Anticancer Drugs: Novel Therapeutic Approaches, Molecular Mechanisms, and Clinical Trials,” Front Pharmacol. 11:529881 (2020); Yang et al., “The rheumatoid arthritis drug auranofin exerts potent anti-lymphoma effect by stimulating TXNRD-mediated ROS generation and inhibition of energy metabolism,” Redox Biol. 75:103245 (2024); Zou et al., “Auranofin induces apoptosis by ROS-mediated ER stress and mitochondrial dysfunction and displayed synergistic lethality with piperlongumine in gastric cancer,” Oncotarget. 6(34):36505-21 (2015)).

Accordingly, there is a need for improved compositions and methods for up-regulating reactive oxygen species. Embodiments of the present disclosure are directed to this and other considerations.

### BRIEF SUMMARY

An exemplary embodiment of the present disclosure provides a compound of formula (I) having the following structure:

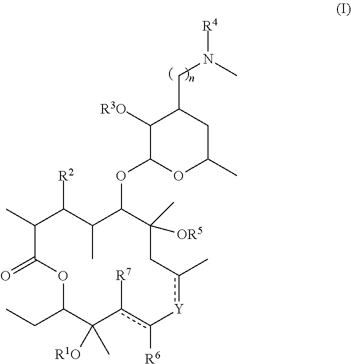

or a stereoisomer, pharmaceutically acceptable salt, oxide, or solvate thereof, wherein
R¹, is a bond, H, or C₁-C₆ alkyl;
R² is oxo or —OR⁸;
R³, R⁵, and R⁶ are each independently H or C₁-C₆ alkyl;
R⁴ is H or C₁-C₆ alkyl, wherein C₁-C₆ alkyl can be substituted with

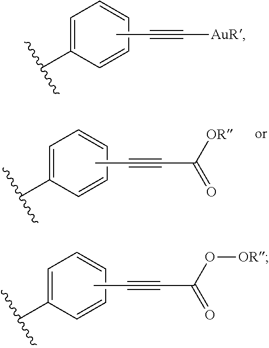

R⁷ is H, hydroxyl, C₁-C₆ alkyl, C₁-C₆ alkoxy, —NR^{a}R^{b},
R⁸ is H, C₁-C₆ alkyl, or

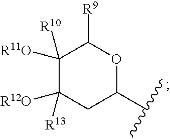

R⁹, R¹¹, R¹², and R¹³ are each independently H or C₁-C₆ alkyl;
R¹⁰ is H, C₁-C₆ alkyl, or

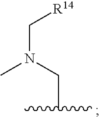

R¹⁴ is H or C₁-C₆ alkyl, wherein C₁-C₆ alkyl can be substituted with

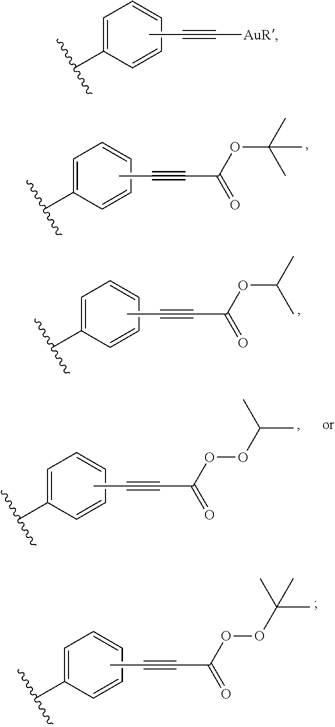

Y is —NR¹⁵CH₂—, C═O, C, C═NR^{c}, or CH₂;
[symbol] is a single or double bond;
R¹⁵ is H or C₁-C₆ alkyl; and
R′ is

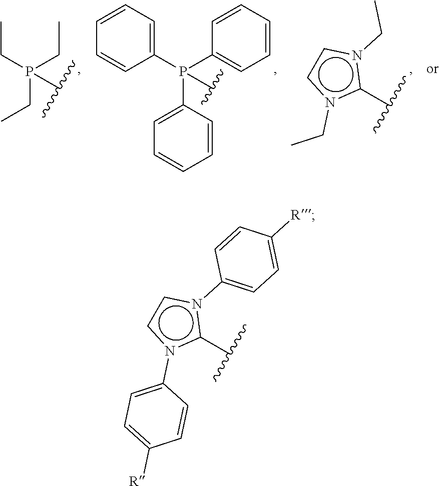

R″ is branched or unbranched C₁₋₆ alkyl;
R′″ is H, C₁₋₆ alkyl, halogen, or —OCH₃
R^{a} H or C₁₋₆ alkyl, wherein C₁₋₆ alkyl can be substituted with oxo;
R^{b} is H or C₁₋₆ alkyl;
R^{c} is H or C₁₋₆ alkyl; or
R¹ and R^{a}, and R^{b} and Re come together with the atoms to which they are attached to form a tricyclic ring with the macrocycle of formula (I); or
Y is a carbon and R¹ and is a bond connected to Y to form a bicyclic ring with the macrocycle of formula (I).

Another exemplary embodiment of the present disclosure provides a compound of formula (I) having the following structure:

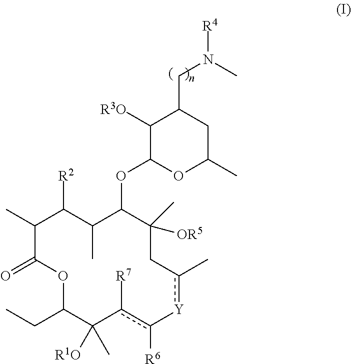

or a stereoisomer, pharmaceutically acceptable salt, oxide, or solvate thereof, wherein
R¹, is a bond, H, or C₁-C₆ alkyl;
R² is oxo or —OR⁸;
R³, R⁵, and R⁶ are each independently H or C₁-C₆ alkyl;
R⁴ is H or C₁-C₆ alkyl, wherein C₁-C₆ alkyl can be substituted with

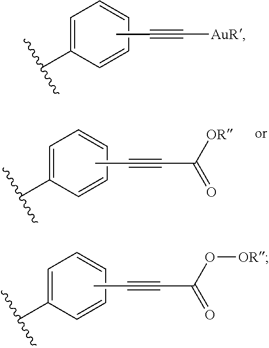

R⁷ is H, hydroxyl, C₁-C₆ alkyl, C₁-C₆ alkoxy, —NR^{a}R^{b};
R⁸ is H, C₁-C₆ alkyl, or

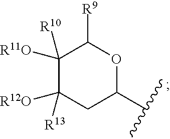

R⁹, R¹¹, R¹², and R¹³ are each independently H or C₁-C₆ alkyl;
R¹⁰ is H, C₁-C₆ alkyl, or

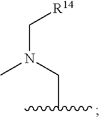

R¹⁴ is H or C₁-C₆ alkyl, wherein C₁-C₆ alkyl can be substituted with

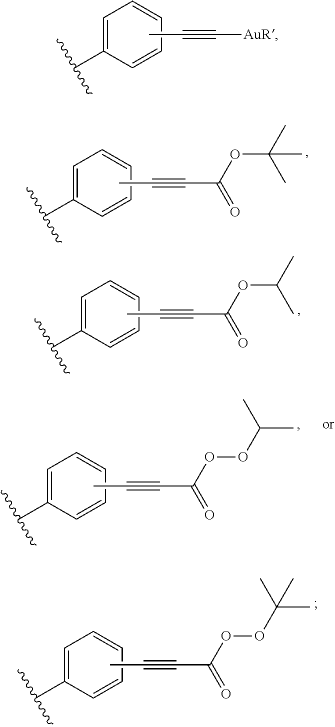

Y is —NR¹⁵CH₂—, C═O, C═NR^{c}, or CH₂;
[symbol] is a single or double bond;
R¹⁵ is H or C₁-C₆ alkyl; and
R′ is

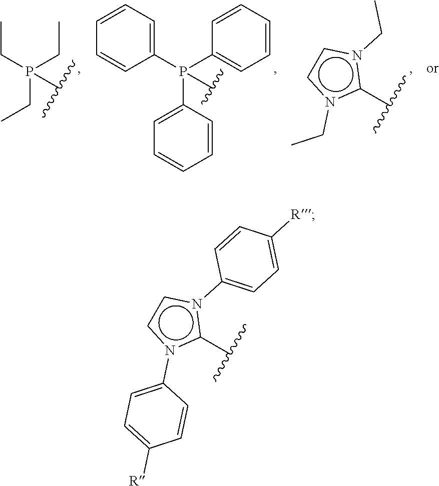

R″ is branched or unbranched C₁₋₆ alkyl;
R″ is H, C₁₋₆ alkyl, halogen, or —OCH₃
R^{a} H or C₁₋₆ alkyl, wherein C₁₋₆ alkyl can be substituted with oxo;
R^{b} is H or C₁₋₆ alkyl;
R^{c} is H or C₁₋₆ alkyl; or
R¹ and R^{a}, and R^{b} and R^{c} come together with the atoms to which they are attached to form a tricyclic ring with the macrocycle of formula (I); or
R¹ and is a bond connected to Y to form a bicyclic ring with the macrocycle of formula (I).

In any of the embodiments disclosed herein, R⁴ can be C₁-C₆ alkyl substituted with

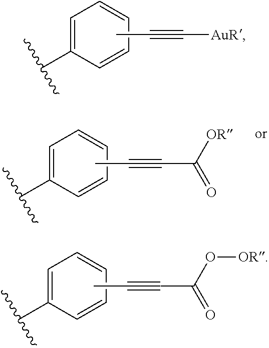

In any of the embodiments disclosed herein, n can be 0.

In any of the embodiments disclosed herein, R² can be —OR⁸, R⁸ can be

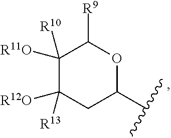

### and R¹⁰ can be H.

In any of the embodiments disclosed herein, R² can be oxo.

In any of the embodiments disclosed herein, n can be 1.

In any of the embodiments disclosed herein, R² can be —OR⁸, R⁸ can be

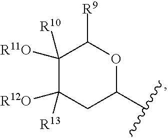

### R¹⁰ can be

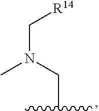

and R¹⁴ can be C₁-C₆ alkyl substituted with

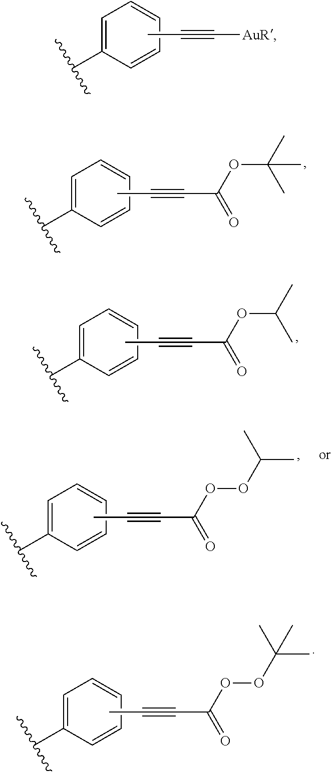

In any of the embodiments disclosed herein, the compound of formula (I) can be selected from the group consisting of

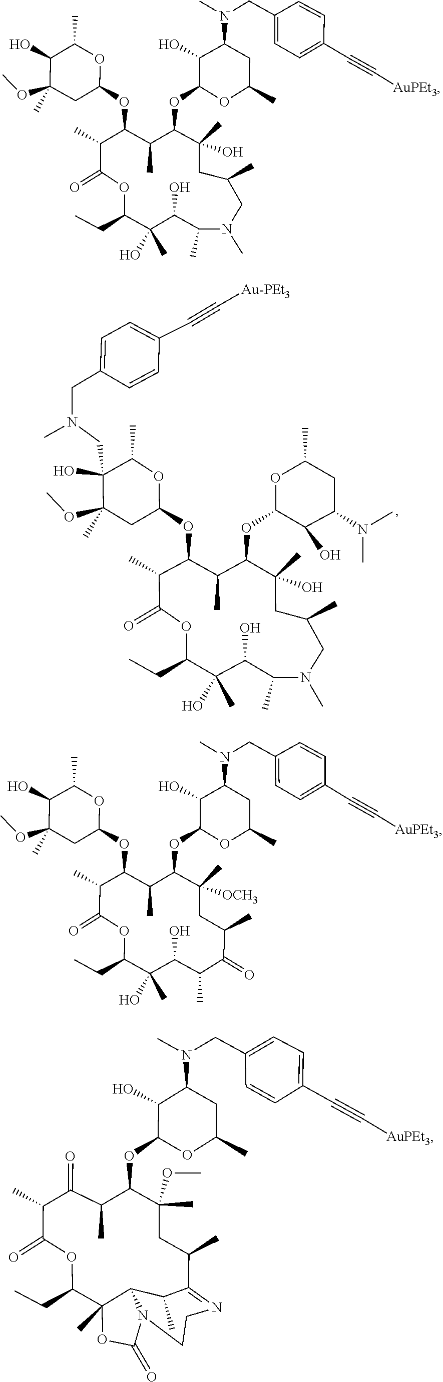

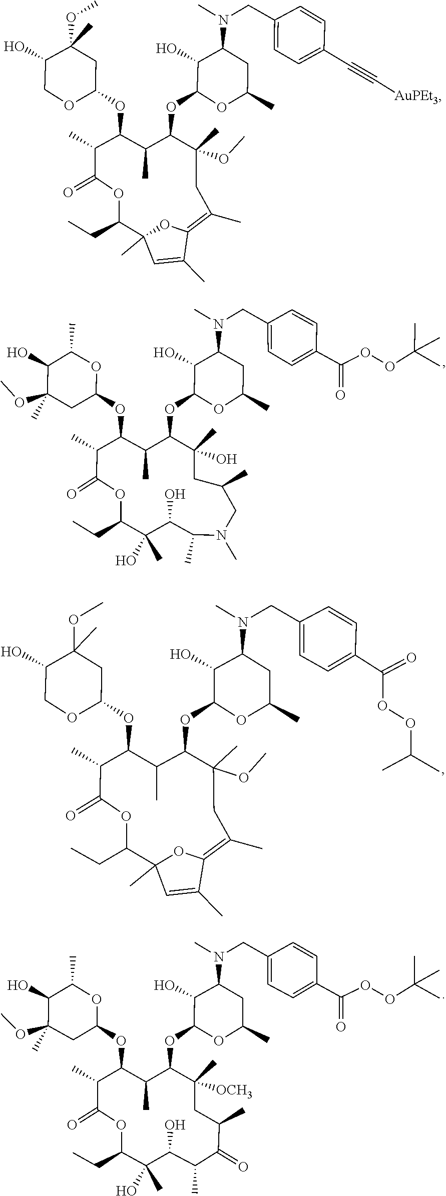

Another exemplary embodiment of the present disclosure provides a method of treating a cancer in a subject. The method can comprise providing a compound as disclosed herein to a subject in need of said treatment.

In any of the embodiments disclosed herein, the cancer can be selected from the group consisting of prostate cancer, lung cancer, liver cancer, or breast cancer.

Another exemplary embodiment of the present disclosure provides a method of up-regulating reactive oxygen species (ROS) in a cancer cell. The method can comprise contacting the cancer cell with a compound as disclosed herein under conditions effective to up-regulate reactive oxygen species in the cell.

In any of the embodiments disclosed herein, the cancer cell can be selected from the group consisting of a prostate, lung, liver, or breast cancer cell.

In any of the embodiments disclosed herein, up-regulating reactive oxygen species in the cancer cell can promote ferroptosis of the cancer cell.

In any of the embodiments disclosed herein, up-regulating reactive oxygen species in the cancer cell can inhibit proliferation of the cancer cell.

Another exemplary embodiment of the present disclosure provides a method of treating malaria in a subject. The method can comprise providing a compound as disclosed herein to a subject in need of said treatment.

Another exemplary embodiment of the present disclosure provides a composition comprising a compound as disclosed herein and a carrier.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIGS. 1A-1E show that DW-678 induced lipid peroxidation in MDA-MB-231 cells, in accordance with an exemplary embodiment of the present disclosure. MDA-MB-231 (1×10⁶) cell lines were seeded for 24 h and treated with various concentrations of DW-678 (5 μM (FIG. 1A), 15 μM (FIG. 1B), 30 μM μM (FIG. 1C)), DW-763 (15 μM (FIG. 1D)), Azithromycin (15 μM (FIG. 1E)) or DMSO. BODIPY 581/591-C11 probe (2 μM) was added during treatment and incubated for 6-h. Fluorescence was detected by FACS, and mean fluorescence intensities of triplicate samples were compiled in part.

FIGS. 2A-2F show that Deferiprone (DFP), Ferrostatin-1 (Fer1) and N-acetylcysteine (NAC) inhibited lipid peroxidation in MDA-MB-231 cells treated with DW-678, in accordance with an exemplary embodiment of the present disclosure. MDA-MB-231 (1×10⁶) cell lines were seeded for 24 h and treated with various concentrations of DW-678 (15 μM (FIG. 2A) and 30 μM (FIG. 2B)), DW-678 (30 μM)+DFP (50 μM) (FIG. 2C), DW-678 (30 M)+Fer1 (1 μM) (FIG. 2D), DW-678 (30 μM)+NAC (2.5 mM) (FIG. 2E), or DMSO. FIG. 2F shows MDA-MB-231 (1×10⁶) cell lines treated with DW-678 (15 μM and 30 μM), DW-678 (30 μM)+DFP (50 μM), DW-678 (30 μM)+Fer1 (1 μM), DW-678 (30 μM)+NAC (2.5 mM), or DMSO. BODIPY 581/591-C11 probe (2 μM) was added during treatment and incubated for 6-h. Fluorescence was detected by FACS, and mean fluorescence intensities of triplicate samples were compiled in part.

FIGS. 3A-3F show that DW-678 triggered ROS generation in MDA-MB-231 and MCF-7 cells, in accordance with an exemplary embodiment of the present disclosure. MDA-MB-231 and MCF-7 cell lines were seeded for 24-h, treated with 10 μM H2DCF-DA, and incubated for 45 min. After incubation, cells were washed with base medium (without FBS), exposed to various treatments DW-678, DW-763, AZM and DW-475, and incubated for 1-h (FIG. 3A, FIG. 3F), 2-h (FIG. 3B, FIG. 3E) and 3-h (FIG. 3C, FIG. 3D). The intensity of DCF-fluorescence (ROS level) was determined using iTecan Microplate Reader using the following wavelength parameters: Excitation: 485 nm and Emission: 535 nm.

FIG. 4 shows that ROS generation is dependent on covalent connection to macrolide moiety as combination of AZM and DW-763 are not as efficient as DW-678 in triggering ROS generation in MDC-MB-231 cells, in accordance with an exemplary embodiment of the present disclosure. MDA-MB-231 (1×10⁶) cell lines were seeded for 24 h and treated with various concentrations of DW-678 (30 μM), DW-673 (30 μM), AZM (30 μM), DW-763+AZM (30 μM) or DMSO. H2DCF-DA probe (10 μM) was added during treatment and incubated for 1-h. DCF fluorescence was detected by FACS, and mean DCF fluorescence intensities of triplicate samples were compiled.

FIGS. 5A-5B show that DW-690, DW-691 and DW-854 triggered ROS generation in MCF-7 cells, in accordance with an exemplary embodiment of the present disclosure. MCF-7 (1×10⁶) cell lines were seeded for 24 h and treated with DW-690, DW-854, AZM (30 μM) or DMSO (FIG. 5A), or DW-691, DW-854, AZM (30 μM), or DMSO (FIG. 5B). H2DCF-DA probe (10 μM) was added during treatment and incubated for 1-h. DCF fluorescence was detected by FACS, and mean DCF fluorescence intensities of triplicate samples were compiled.

FIG. 6 shows antimalarial activities of select exemplifying compounds of formula (I), in accordance with an exemplary embodiment of the present disclosure.

### DETAILED DESCRIPTION

Although preferred exemplary embodiments of the disclosure are explained in detail, it is to be understood that other exemplary embodiments are contemplated. Accordingly, it is not intended that the disclosure is limited in its scope to the details of construction and arrangement of components set forth in the following description or illustrated in the drawings. The disclosure is capable of other exemplary embodiments and of being practiced or carried out in various ways. Also, in describing the preferred exemplary embodiments, specific terminology will be resorted to for the sake of clarity.

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

As used in the specification and the appended claims, the singular forms “a,” “an” and “the” include plural referents unless the context clearly dictates otherwise.

Also, in describing the preferred exemplary embodiments, terminology will be resorted to for the sake of clarity. It is intended that each term contemplates its broadest meaning as understood by those skilled in the art and includes all technical equivalents which operate in a similar manner to accomplish a similar purpose.

Ranges can be expressed herein as from “about” or “approximately” one particular value and/or to “about” or “approximately” another particular value. When such a range is expressed, another exemplary embodiment includes from the one particular value and/or to the other particular value.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, member, particle, or method step is present in the composition or article or method, but does not exclude the presence of other compounds, materials, particles, method steps, even if the other such compounds, material, particles, method steps have the same function as what is named.

Mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

The materials described as making up the various members of the invention are intended to be illustrative and not restrictive. Many suitable materials that would perform the same or a similar function as the materials described herein are intended to be embraced within the scope of the invention. Such other materials not described herein can include, but are not limited to, for example, materials that are developed after the time of the development of the invention.

Reference will now be made in detail to exemplary embodiments of the disclosed technology, examples of which are illustrated in the accompanying drawings and disclosed herein. Wherever convenient, the same references numbers will be used throughout the drawings to refer to the same or like parts.

The present disclosure describes a composition of matter comprising macrocycle oxidative stress inducers and their use as anticancer agents that function by up-regulating reactive oxygen species (ROS) and ferroptosis in cancer cells and selectively accumulate within the tumors of the liver, lungs, and breast. The compounds inhibit the viability and proliferation of several cancer cells including prostate, lung, liver, and breast cancers.

An exemplary embodiment of the present disclosure provides a compound of formula (I) having the following structure:

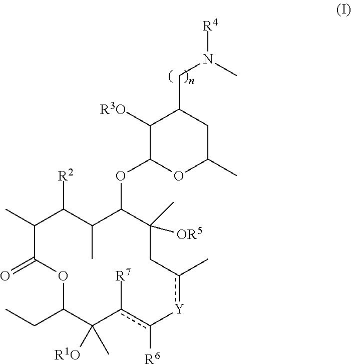

or a stereoisomer, pharmaceutically acceptable salt, oxide, or solvate thereof, wherein
R¹, is a bond, H, or C₁-C₆ alkyl;
R² is oxo or —OR⁸;
R³, R⁵, and R⁶ are each independently H or C₁-C₆ alkyl;
R⁴ is H or C₁-C₆ alkyl, wherein C₁-C₆ alkyl can be substituted with

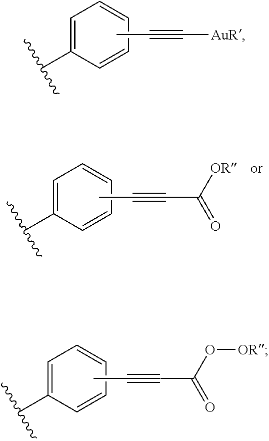

R⁷ is H, hydroxyl, C₁-C₆ alkyl, C₁-C₆ alkoxy, —NR^{a}R^{b},
R⁸ is H, C₁-C₆ alkyl, or

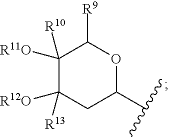

R⁹, R¹¹, R¹², and R¹³ are each independently H or C₁-C₆ alkyl;
R¹⁰ is H, C₁-C₆ alkyl, or

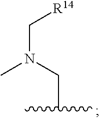

R¹⁴ is H or C₁-C₆ alkyl, wherein C₁-C₆ alkyl can be substituted with

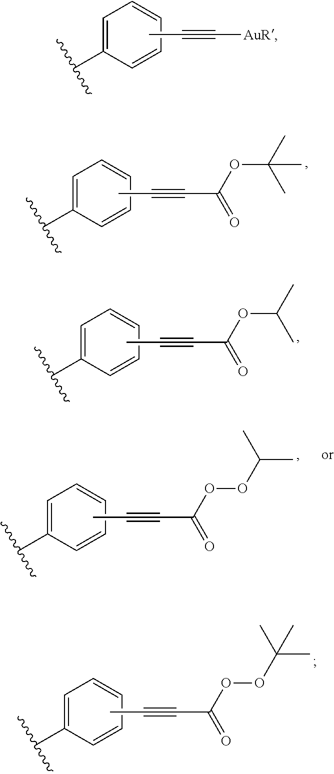

Y is —NR¹⁵CH₂, C, C═O, C═NR^{c}, or CH₂;
[symbol] is a single or double bond;
R¹⁵ is H or C₁-C₆ alkyl; and
R′ is

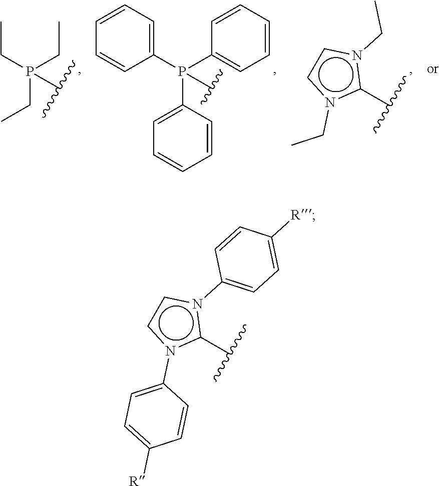

R″ is branched or unbranched C₁₋₆ alkyl;
R″ is H, C₁₋₆ alkyl, halogen, or —OCH₃
R^{a} H or C₁₋₆ alkyl, wherein C₁₋₆ alkyl can be substituted with oxo;
R^{b} is H or C₁₋₆ alkyl;
R^{c} is H or C₁₋₆ alkyl; or
R¹ and R^{a}, and R^{b} and Re come together with the atoms to which they are attached to form a tricyclic ring with the macrocycle of formula (I); or
R¹ and is a bond connected to Y to form a bicyclic ring with the macrocycle of formula (I).

Another exemplary embodiment of the present disclosure provides a compound of formula (II) or formula (III):

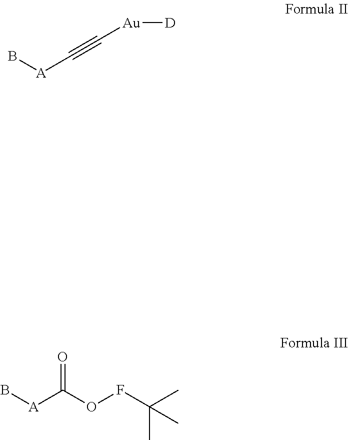

wherein
A is C₁₋₆ alkyl, optionally substituted with unsaturation and aryl groups independently connected at the ortho, meta or para position;
B is chosen from 13-membered, bicyclic-13, 14-membered, tricyclic-5, 6, 14-membered, 14-membered, 15-membered macrolides including but not limited to

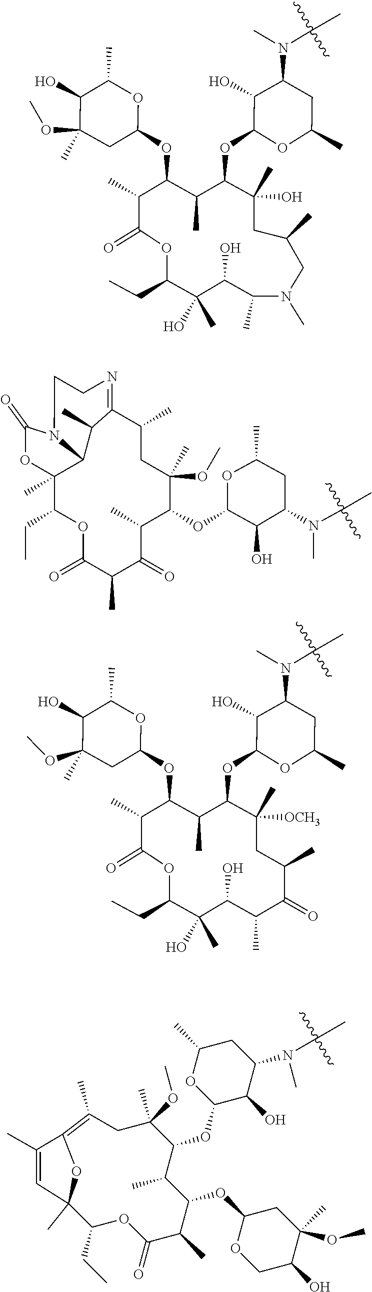

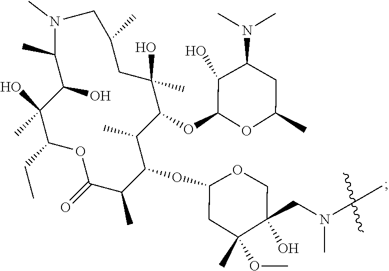 [SMILES: *N(C)C[C@]1(O)CO[C@@H](O[C@H]2[C@H](C)[C@@H](O[C@@H]3O[C@H](C)C[C@H](N(C)C)[C@H]3O)[C@](C)(O)C[C@@H](C)CN(C)[C@H](C)[C@@H](O)[C@](C)(O)[C@@H](CC)OC(=O)[C@@H]2C)C[C@@]1(C)OC]

wherein the macrolides are optionally connected to A at the desosamine or cladinose sugar;
D is a ligand chosen from but not limited to

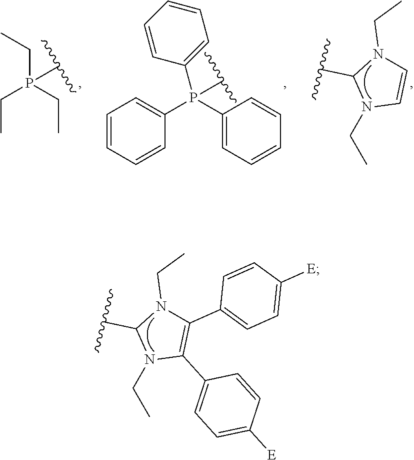

E is independently H, C₁₋₆ alkyl, halogen, and OMe;
F is O or absent.

As used above, and throughout the description herein, the following terms, unless otherwise indicated, shall be understood to have the following meanings. If not defined otherwise herein, all technical and scientific terms used herein have the same meaning as is commonly understood by one of ordinary skill in the art to which this technology belongs. In the event that there is a plurality of definitions for a term herein, those in this section prevail unless stated otherwise.

The term “alkyl” means an aliphatic hydrocarbon group which may be straight or branched having about 1 to about 12 carbon atoms in the chain. Particular alkyl groups have 1 to about 6 carbon atoms in the chain. Branched means that one or more lower alkyl groups such as methyl, ethyl or propyl are attached to a linear alkyl chain. Exemplary alkyl groups include methyl, ethyl, n-propyl, i-propyl, n-butyl, t-butyl, n-pentyl, and 3-pentyl. The term “alkylene” refers to a divalent group formed from an alkane by removal of two hydrogen atoms. Exemplary alkylene groups include, but are not limited to, methylene, ethylene, propylene, and other divalent groups derived from the alkanes described above.

The term “alkenyl” means an aliphatic hydrocarbon group containing a carbon-carbon double bond and which may be straight or branched having about 2 to about 6 carbon atoms in the chain. Particular alkenyl groups have 2 to about 4 carbon atoms in the chain. Branched means that one or more lower alkyl groups such as methyl, ethyl, or propyl are attached to a linear alkenyl chain. Exemplary alkenyl groups include ethenyl, propenyl, n-butenyl, and i-butenyl. The term “alkenyl” may also refer to a hydrocarbon chain having 2 to 6 carbons containing at least one double bond and at least one triple bond.

The term “aryl” means an aromatic monocyclic or multicyclic ring system of 6 to about 14 carbon atoms, preferably of 6 to about 10 carbon atoms. Representative aryl groups include phenyl and naphthyl. The term “arylene” refers to a group obtained by removal of a hydrogen atom from an aryl group. Non-limiting examples of arylene include phenylene and naphthylene,

The term “heteroaryl” means an aromatic monocyclic or multicyclic ring system of about 5 to about 14 ring atoms, preferably about 5 to about 10 ring atoms, in which one or more of the atoms in the ring system is/are element(s) other than carbon, for example, nitrogen, oxygen, or sulfur. In the case of multicyclic ring system, only one of the rings needs to be aromatic for the ring system to be defined as “Heteroaryl”. Preferred heteroaryls contain about 5 to 6 ring atoms. The prefix aza, oxa, thia, or thio before heteroaryl means that at least a nitrogen, oxygen, or sulfur atom, respectively, is present as a ring atom. A nitrogen atom of a heteroaryl is optionally oxidized to the corresponding N-oxide. Representative heteroaryls include pyridyl, 2-oxo-pyridinyl, pyrimidinyl, pyridazinyl, pyrazinyl, triazinyl, furanyl, pyrrolyl, thiophenyl, pyrazolyl, imidazolyl, oxazolyl, isoxazolyl, thiazolyl, isothiazolyl, triazolyl, oxadiazolyl, thiadiazolyl, tetrazolyl, indolyl, isoindolyl, benzofuranyl, benzothiophenyl, indolinyl, 2-oxoindolinyl, dihydrobenzofuranyl, dihydrobenzothiophenyl, indazolyl, benzimidazolyl, benzooxazolyl, benzothiazolyl, benzoisoxazolyl, benzoisothiazolyl, benzotriazolyl, benzo[1,3]dioxolyl, quinolinyl, isoquinolinyl, quinazolinyl, cinnolinyl, pthalazinyl, quinoxalinyl, 2,3-dihydro-benzo[1,4]dioxinyl, benzo[1,2,3]triazinyl, benzo[1,2,4]triazinyl, 4H-chromenyl, indolizinyl, quinolizinyl, 6aH-thieno[2,3-d]imidazolyl, 1H-pyrrolo[2,3-b]pyridinyl, imidazo[1,2-a]pyridinyl, pyrazolo[1,5-a]pyridinyl, [1,2,4]triazolo[4,3-a]pyridinyl, [1,2,4]triazolo[1,5-a]pyridinyl, thieno[2,3-b]furanyl, thieno[2,3-b]pyridinyl, thieno[3,2-b]pyridinyl, furo[2,3-b]pyridinyl, furo[3,2-b]pyridinyl, thieno[3,2-d]pyrimidinyl, furo[3,2-d]pyrimidinyl, thieno[2,3-b]pyrazinyl, imidazo[1,2-a]pyrazinyl, 5,6,7,8-tetrahydroimidazo[1,2-a]pyrazinyl, 6,7-dihydro-4H-pyrazolo[5,1-c][1,4]oxazinyl, 2-oxo-2,3-dihydrobenzo[d]oxazolyl, 3,3-dimethyl-2-oxoindolinyl, 2-oxo-2,3-dihydro-1H-pyrrolo[2,3-b]pyridinyl, benzo[c][1,2,5]oxadiazolyl, benzo[c][1,2,5]thiadiazolyl, 3,4-dihydro-2H-benzo[b][1,4]oxazinyl, 5,6,7,8-tetrahydro-[1,2,4]triazolo[4,3-a]pyrazinyl, [1,2,4]triazolo[4,3-a]pyrazinyl, 3-oxo-[1,2,4]triazolo[4,3-a]pyridin-2 (3H)-yl, and the like. The term “heteroarylene” refers to a group obtained by removal of a hydrogen atom from a heteroaryl group. Exemplary heteroarylene groups include, but are not limited to, groups derived from the heteroaryl groups described above.

As used herein, “heterocyclyl” refers to a stable 3- to 18-membered ring (radical) which consists of carbon atoms and from one to five heteroatoms selected from the group consisting of nitrogen, oxygen and sulfur. For purposes of this application, the heterocycle may be a monocyclic, or a polycyclic ring system, which may include fused, bridged, or spiro ring systems; and the nitrogen, carbon, or sulfur atoms in the heterocycle may be optionally oxidized; the nitrogen atom may be optionally quaternized; and the ring may be partially or fully saturated. For purposes of this application, when the heterocycle is a polycyclic ring system, one of the rings can be an aromatic ring and another one is non-aromatic. Examples of heterocycles include, without limitation, azepinyl, azocanyl, pyranyl dioxanyl, dithianyl, 1,3-dioxolanyl, tetrahydrofuryl, dihydropyrrolidinyl, decahydroisoquinolyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, 2-oxoazepinyl, oxazolidinyl, oxiranyl, piperidinyl, piperazinyl, 4-piperidonyl, pyrrolidinyl, pyrazolidinyl, thiazolidinyl, tetrahydropyranyl, thiamorpholinyl, thiamorpholinyl sulfoxide, and thiamorpholinyl sulfone. Further heterocycles and heteroaryls are described in Katritzky et al., eds., Comprehensive Heterocyclic Chemistry: The Structure, Reactions, Synthesis and Use of Heterocyclic Compounds, Vol. 1-8, Pergamon Press, N.Y. (1984).

The term “halogen” means fluoro, chloro, bromo, or iodo.

The term “substituted” or “substitution” of an atom means that one or more hydrogen on the designated atom is replaced with a selection from the indicated group, provided that the designated atom's normal valency is not exceeded.

“Unsubstituted” atoms bear all of the hydrogen atoms dictated by their valency. When a substituent is keto (i.e., ═O), then two hydrogens on the atom are replaced. Combinations of substituents and/or variables are permissible only if such combinations result in stable compounds; by “stable compound” or “stable structure” is meant a compound that is sufficiently robust to survive isolation to a useful degree of purity from a reaction mixture, and formulation into an efficacious therapeutic agent.

The term “optionally substituted” is used to indicate that a group may have a substituent at each substitutable atom of the group (including more than one substituent on a single atom), provided that the designated atom's normal valency is not exceeded and the identity of each substituent is independent of the others. Up to three H atoms in each residue are replaced with alkyl, halogen, haloalkyl, hydroxy, loweralkoxy, carboxy, carboalkoxy (also referred to as alkoxycarbonyl), carboxamido (also referred to as alkylaminocarbonyl), cyano, carbonyl, nitro, amino, alkylamino, dialkylamino, mercapto, alkylthio, sulfoxide, sulfone, acylamino, amidino, phenyl, benzyl, heteroaryl, phenoxy, benzyloxy, or heteroaryloxy.

Compounds described herein may contain one or more asymmetric centers and may thus give rise to enantiomers, diastereomers, and other stereoisomeric forms. Each chiral center may be defined, in terms of absolute stereochemistry, as (R)- or (S)-. This technology is meant to include all such possible isomers, as well as mixtures thereof, including racemic and optically pure forms. Optically active (R)- and (S)-, (−)- and (+)—, or (D)- and (L)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

This technology also envisions the “quaternization” of any basic nitrogen-containing groups of the compounds disclosed herein. The basic nitrogen can be quaternized with any agents known to those of ordinary skill in the art including, for example, lower alkyl halides, such as methyl, ethyl, propyl and butyl chloride, bromides and iodides; dialkyl sulfates including dimethyl, diethyl, dibutyl and diamyl sulfates; long chain halides such as decyl, lauryl, myristyl and stearyl chlorides, bromides and iodides; and aralkyl halides including benzyl and phenethyl bromides. Water or oil-soluble or dispersible products may be obtained by such quaternization.

In the characterization of some of the substituents, it is recited that certain substituents may combine to form rings. Unless stated otherwise, it is intended that such rings may exhibit various degrees of unsaturation (from fully saturated to fully unsaturated), may include heteroatoms and may be substituted with lower alkyl or alkoxy.

The term “pharmaceutically acceptable salts” means the relatively non-toxic, inorganic, and organic acid addition salts, and base addition salts, of compounds of the present disclosure. These salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulphamates, malonates, salicylates, propionates, methylene-bis-b-hydroxynaphthoates, gentisates, isethionates, di-p-toluoyltartrates, methane-sulphonates, ethanesulphonates, benzenesulphonates, p-toluenesulphonates, cyclohexylsulphamates and quinateslaurylsulphonate salts, and the like (see, for example, Berge et al., “Pharmaceutical Salts,” J. Pharm. Sci., 66:1-9 (1977) and Remington's Pharmaceutical Sciences, 17th ed., Mack Publishing Company, Easton, Pa., 1985, p. 1418). Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred. Suitable inorganic base addition salts are prepared from metal bases which include, for example, sodium hydride, sodium hydroxide, potassium hydroxide, calcium hydroxide, aluminum hydroxide, lithium hydroxide, magnesium hydroxide, and zinc hydroxide. Suitable amine base addition salts are prepared from amines which have sufficient basicity to form a stable salt, and preferably include those amines which are frequently used in medicinal chemistry because of their low toxicity and acceptability for medical use, such as ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylarnine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, dicyclohexylamine, and the like.

The term “solvate” refers to a compound of formula (I) in the solid state, wherein molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions.

The term “pharmaceutically acceptable” means it is, within the scope of sound medical judgement, suitable for use in contact with the cells of humans and lower animals without undue toxicity, irritation, allergic response and the like, and are commensurate with a reasonable benefit/risk ratio.

In any of the embodiments of formula (I) disclosed herein, R⁴ can be C₁-C₆ alkyl substituted with

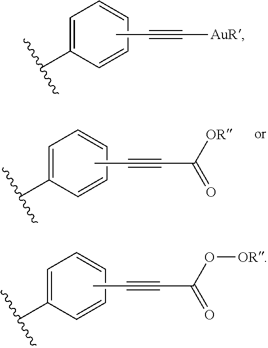

In some embodiments of formula (I), n is 0.

In some embodiments of formula (I), R² is —OR⁸, R⁸ is

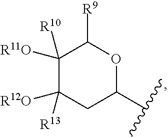

### and R¹⁰ is H.

In some embodiments of formula (I), R² is oxo.

In any of the embodiments of formula (I) disclosed herein, n can be 1. In some embodiments of formula (I), R² is —OR⁸, R⁸ is

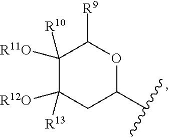

### R¹⁰ is

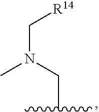

and R¹⁴ is C₁-C₆ alkyl substituted with

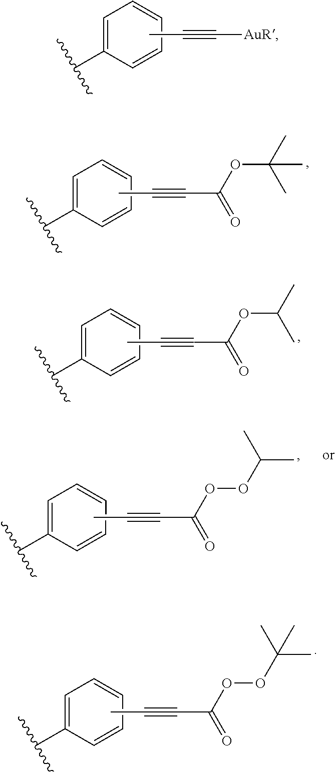

In some embodiments of formula (I), Y can be a carbon and R¹ is a bond connected to Y [a carbon] to form a bicyclic ring with the macrocycle of formula (I). In some embodiments, where R¹ is a bond connected to Y to form a bicyclic ring with the macrocycle of formula (I), the compound of formula (I) has the following structure:

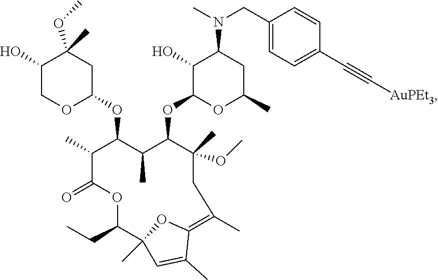

In some embodiments of formula (I), R¹ and R^{a}, and R^{b} and R^{c} come together with the atoms to which they are attached to form a tricyclic ring with the macrocycle of formula (I), such that the compound of formula (I) has the following structure:

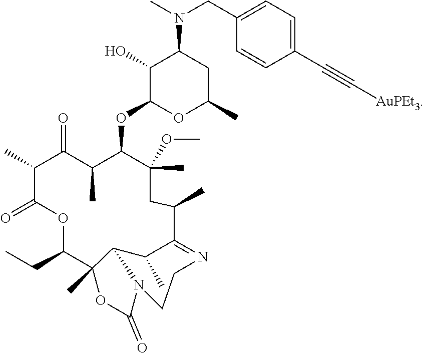

In any of the embodiments disclosed herein, the compound of formula (I) can be selected from the group consisting of

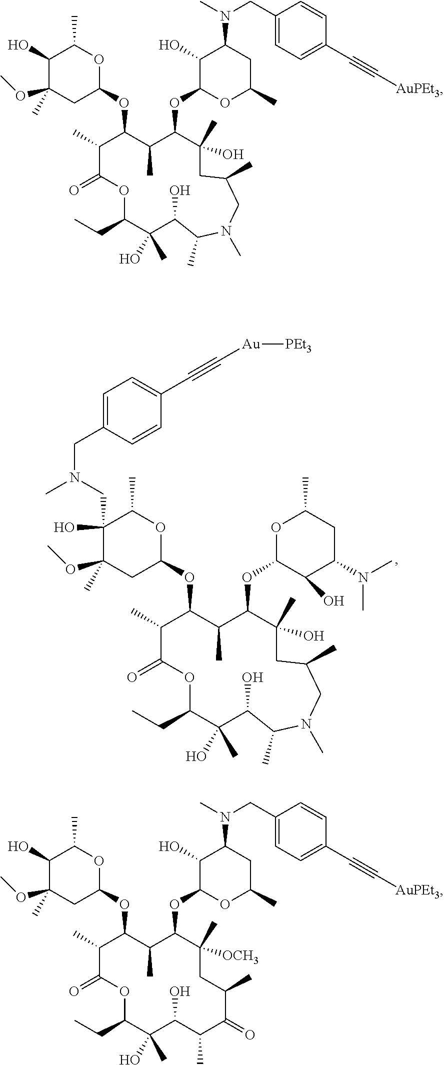

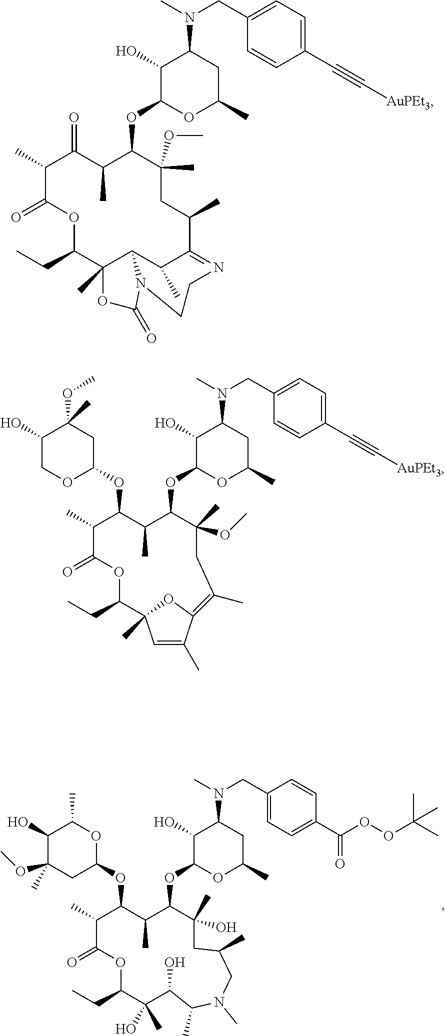

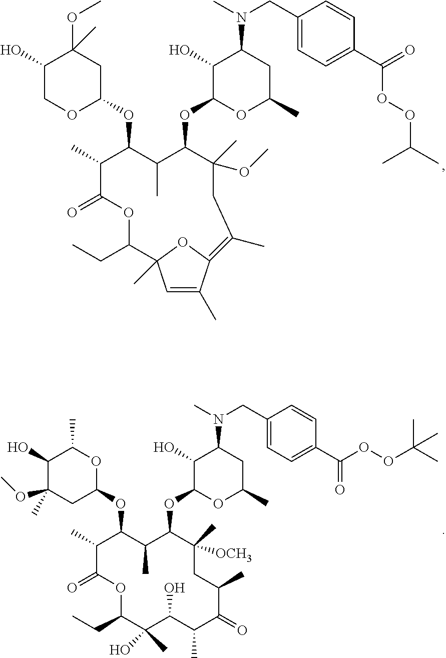

Another exemplary embodiment of the present disclosure provides a method of treating a cancer in a subject. The method can comprise providing a compound as disclosed herein to a subject in need of said treatment.

The term “method of treating” means amelioration or relief from the symptoms and/or effects associated with the disorders described herein. As used herein, reference to “treatment” of a patient is intended to include prophylaxis.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

A “subject” or “patient” or “individual” or “animal”, as used herein, refers to humans, veterinary animals (e.g., cats, dogs, cows, horses, sheep, pigs, etc.) and experimental animal models of diseases (e.g., mice, rats). In a preferred embodiment, the subject is a human.

As used herein the term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

While it may be possible for compounds of formula (I), formula (II), and formula (III) to be administered as raw chemicals, it can be preferable to present them as a part of a pharmaceutical composition. Accordingly, another aspect of the present invention is a pharmaceutical composition containing a therapeutically effective amount of the compound of formula (I), formula (II), or formula (III), or a pharmaceutically acceptable salt thereof, and a pharmaceutically acceptable carrier.

The term “pharmaceutically acceptable dosage forms” means dosage forms of the compound of the invention, and includes, for example, tablets, dragees, powders, elixirs, syrups, liquid preparations, including suspensions, sprays, inhalants tablets, lozenges, emulsions, solutions, granules, capsules, and suppositories, as well as liquid preparations for injections, including liposome preparations. Techniques and formulations generally may be found in Remington's Pharmaceutical Sciences, Mack Publishing Co., Easton, Pa., latest edition.

The compounds and pharmaceutical compositions of the present disclosure can be particularly useful for the treatment of cancer. As used herein, the term “cancer” refers to a cellular disorder characterized by uncontrolled or disregulated cell proliferation, decreased cellular differentiation, inappropriate ability to invade surrounding tissue, and/or ability to establish new growth at ectopic sites. The term “cancer” includes, but is not limited to, solid tumors and bloodborne tumors. The term “cancer” encompasses diseases of skin, tissues, organs, bone, cartilage, blood, and vessels. The term “cancer” further encompasses primary and metastatic cancers.

In any of the embodiments disclosed herein, the cancer can be, without limitation, selected from prostate cancer, lung cancer, liver cancer, or breast cancer.

In practicing the method of the present disclosure, agents suitable for treating a subject can be administered using any method standard in the art. The agents, in their appropriate delivery form, can be administered orally, intradermally, intramuscularly, intraperitoneally, intravenously, subcutaneously, or intranasally. The compositions of the present disclosure may be administered alone or with suitable pharmaceutical carriers, and can be in solid or liquid form, such as tablets, capsules, powders, solutions, suspensions, or emulsions.

Another exemplary embodiment of the present disclosure provides a method of up-regulating reactive oxygen species (ROS) in a cancer cell. The method can comprise contacting the cancer cell with a compound as disclosed herein under conditions effective to up-regulate reactive oxygen species in the cell.

In any of the embodiments disclosed herein, the cancer cell can be, without limitation, selected from a prostate, lung, liver, or breast cancer cell. In any of the embodiments disclosed herein, up-regulating reactive oxygen species in the cancer cell can promote ferroptosis of the cancer cell. In any of the embodiments disclosed herein, up-regulating reactive oxygen species in the cancer cell can inhibit proliferation of the cancer cell.

Upregulating reactive oxygen species (ROS) is a way many antimalarial treatments kill Plasmodium parasites. Some antimalarial agents, including artemisinin, work by inducing ROS which is toxic to plasmodium cells due to the presence of high concentration of free ferrous iron and/or iron-rich heme by-products of hemoglobin digestion by the parasite (Rosenthal MR, Ng CL, “Plasmodium falciparum Artemisinin Resistance: The Effect of Heme, Protein Damage, and Parasite Cell Stress Response,” ACS Infect Dis. 6(7):1599-1614 (2020)). Another exemplary embodiment of the present disclosure provides a method of treating malaria in a subject. The method can comprise providing a compound as disclosed herein to a subject in need of said treatment.

Another exemplary embodiment of the present disclosure provides a composition comprising a compound as disclosed herein and a carrier.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

### EXAMPLES

The following Examples are presented to illustrate various aspects of the present disclosure, but are by no means intended to limit its scope.

### Example 1—Synthesis of Exemplifying Compounds

### Synthesis of Macrolide—Gold Conjugate

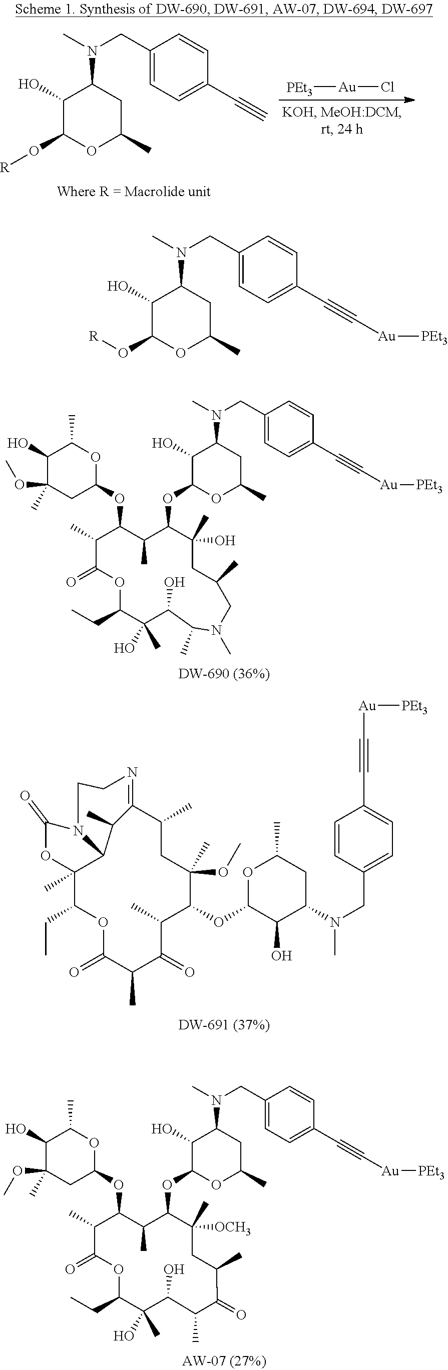

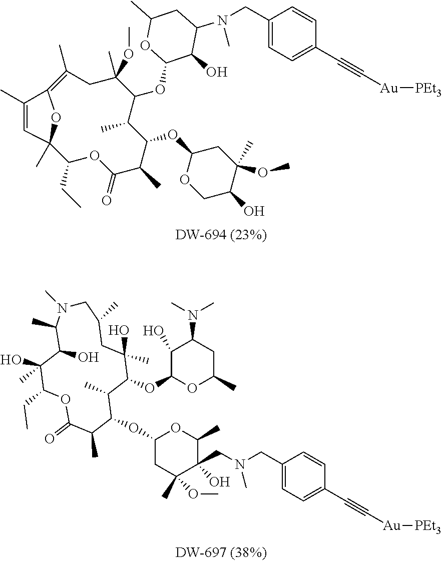

### Synthesis of Macrolide—Endoperoxide Conjugate

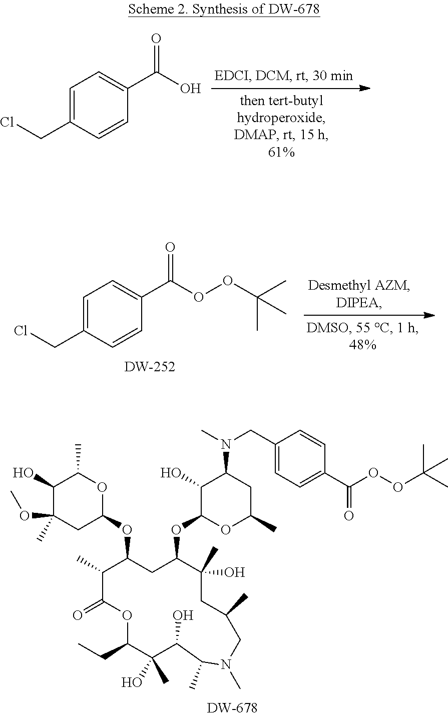 [SMILES: O=C(O)c1ccc(CCl)cc1 ; CC(C)(C)OOC(=O)c1ccc(CCl)cc1 ; CC[C@H]1OC(=O)[C@H](C)[C@@H](O[C@H]2C[C@@](C)(OC)[C@@H](O)[C@H](C)O2)C[C@@H](O[C@@H]2O[C@H](C)C[C@H](N(C)Cc3ccc(C(=O)OOC(C)(C)C)cc3)[C@H]2O)[C@](C)(O)C[C@@H](C)CN(C)[C@H](C)[C@@H](O)[C@]1(C)O]

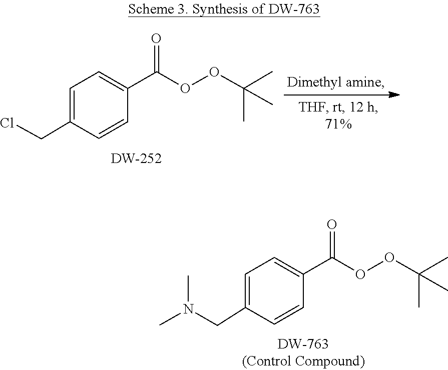 [SMILES: CC(C)(C)OOC(=O)c1ccc(CCl)cc1 ; CN(C)Cc1ccc(C(=O)OOC(C)(C)C)cc1]

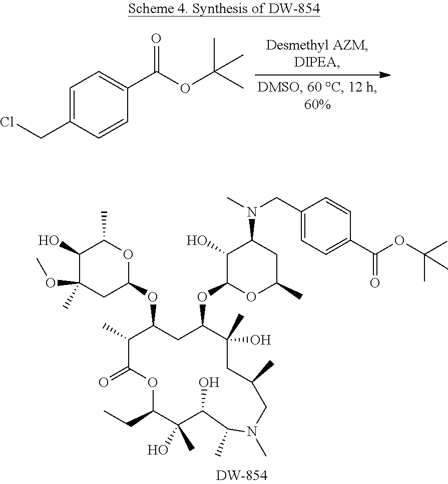 [SMILES: CC(C)(C)OC(=O)c1ccc(CCl)cc1 ; CC[C@H]1OC(=O)[C@H](C)[C@@H](O[C@H]2C[C@@](C)(OC)[C@@H](O)[C@H](C)O2)C[C@@H](O[C@@H]2O[C@H](C)C[C@H](N(C)Cc3ccc(C(=O)OC(C)(C)C)cc3)[C@H]2O)[C@](C)(O)C[C@@H](C)CN(C)[C@H](C)[C@@H](O)[C@]1(C)O]

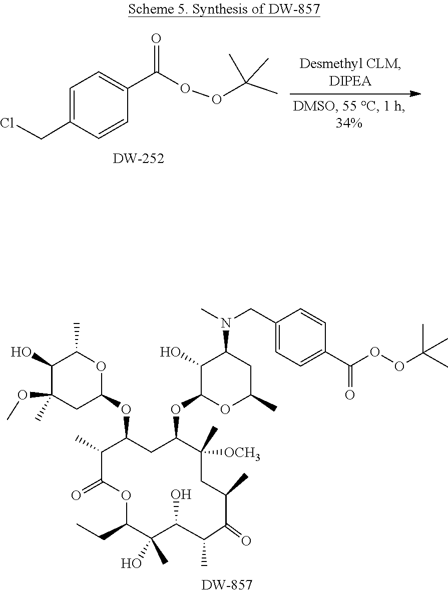

### Example 2—Experimental Procedure and Characterizations

### Experimental Procedure and Characterization for Macrolide—Gold Conjugate

### General Procedure

Macrolide Alkyne (1 equiv.), potassium hydroxide (KOH, 1.2 equiv.), anhydrous methanol (2 mL), and anhydrous DCM (2 mL) were mixed under an argon atmosphere for 15 min. Then Chloro(triethylphosphine) gold (I) (1 equiv.) was added, and the mixture was stirred in the dark 15-24 h at room temperature. TLC indicated the completion of the reaction. The reaction mixture was quenched with water (10 mL), extracted with CH₂Cl₂ (2×20 mL), and the organic layer was sequentially washed water (15 mL), and brine (10 mL). The organic layer was dried over Na₂SO₄, and then filtered. The solvent was removed under reduced pressure to obtain a dark solid. The crude material was purified preparative TLC, using Acetone/Hexanes/MeOH: 3:4:0.2 to afford Macrolide—Gold Conjugate series of compounds as a white solid.

### DW-690

Following the general procedure using AZM Alkyne (1.450 g), Chloro(triethylphosphine) gold (I) (600 mg) and potassium hydroxide (11.5 mg) gave DW-690 (720 mg, 36% yield) as a white solid; ¹H NMR (400 MHZ, CDCl₃) δ 7.41 (d, J=8.3 Hz, 2H), 7.15 (d, J=8.3 Hz, 2H), 5.09 (d, J=4.7 Hz, 1H), 4.66 (d, J=10.4 Hz, 1H), 4.39 (d, 1H), 4.24 (s, 1H), 3.99 (m, 1H), 3.74-3.57 (m, 3H), 3.48-3.23 (m, 3H), 3.10 (s, 3H), 2.98 (d, J=7.8 Hz, 2H), 2.87 (m, 1H), 2.53 (m, 1H), 2.33 (d, J=14.9 Hz, 1H), 2.20 (S, 3H), 2.12-1.89 (m, 4H), 1.85-1.73 (s, 8H), 1.32-0.97 (m, 49H), 0.91-0.84 (m, 8H). ¹³C NMR (101 MHz, CDCl₃) δ 168.2, 142.8, 129.5, 128.7, 127.5, 106, 103, 94, 84, 83, 78, 74, 73, 72, 70, 68, 65, 64, 62, 57, 54, 50, 49, 48, 45, 43, 42, 41 37, 36, 34, 29, 28, 27, 26, 22, 21, 18, 16, 14, 11, 9, 7. HRMS (EI) m/z Calcd, for C52H91AuN2O12P [M+H]⁺: 1163.5975, found: 1163.7369.

### DW-691

Following the general procedure using Macrolide Alkyne (100 mg), Chloro(triethylphosphine) gold (I) (45 mg) and potassium hydroxide (10 mg) gave DW-691 (54 mg, 37% yield) as a white solid; ¹H NMR (400 MHz, CDCl₃) δ 7.39 (d, J=8.4 Hz, 2H), 7.09 (d, J=6.2 Hz, 2H), 4.95 (d, J=10.6 Hz, 1H), 4.21 (m, 1H), 3.94 (d, 1H), 3.83-3.62 (m, 4H), 3.50 (m, 2H), 3.30 (m, 2H), 3.06-2.93 (m, 1H), 2.65 (s, 3H), 2.66-2.52 (m, 2H), 2.13 (s, 3H), 1.83-1.68 (m, 9H), 1.45 (s, 3H), 1.34-1.11 (m, 26H), 1.07-0.96 (d, J=7.0 Hz, 3H), 0.82 (t, J=7.5 Hz, 3H). ¹³C NMR (176 MHz, CDCl₃) δ 178, 167, 159, 136,130, 128, 124, 118, 109, 94, 83, 78, 77, 74, 73, 71, 70, 68, 65, 64, 65, 62, 57, 53, 45.7, 45, 42, 38, 37, 36, 29, 28, 27, 26, 22, 18, 16, 14, 11, 9, 7. HRMS (EI) m/z Calcd, for C₄₇H₇₅AuN₃O₉P [M+H]⁺: 1053.4906, found: 1053.4910.

### DW-694

Following the general procedure using Macrolide e Alkyne (100 mg), Chloro(triethylphosphine) gold (I) (43 mg) and potassium hydroxide (8 mg) gave DW-694 (31 mg, 23% yield) as a white solid; ¹H NMR (400 MHZ, CDCl₃) δ 7.91 (d, J=8.0 Hz, 2H), 7.41 (d, J=8.0 Hz, 2H), 5.69 (s, 1H), 5.0 (d, J=10.8 Hz, 1H), 4.84 (d, J=4.1 Hz, 1H), 4.40-4.34 (d, J=7.2 Hz, 1H), 4.08 (d, J=10.0 Hz, 1H), 3.93 (m, 1H), 3.80 (d, J=13.7 Hz, 1H), 3.59 (d, J=13.5 Hz, 1H), 3.44 (d, J=8.6 Hz, 2H), 3.42 (m, 1H), 3.18 (s, 3H), 3.14 (s, 3H), 2.97 (m, 2H), 2.71-2.62 (m, 1H), 2.59 (s, 3H), 2.30 (d, J=14.9 Hz, 1H), 2.22 (s, 3H), 2.04 (s, 6H), 1.76-1.65 (m, 2H), 1.52-1.34 (m, 4H), 1.31-1.15 (m, 26H), 1.11 (d, J=7.0 Hz, 3H), 0.98 (d, J=4.2 Hz, 3H), 0.87 (d, J=4.1 Hz, 6H). ¹³C NMR (101 MHZ, CDCl₃) δ 175.6 154.3, 135.6, 129.8, 103.8, 96.0, 87.7, 84.3, 81.3, 80.5, 78.1, 72.5, 71.2, 68.8, 65.4, 65.0, 49.4, 47.4, 45.5, 40.5, 39.2, 36.9, 34.9, 29.0, 25.8, 24.6, 24.1, 21.6, 21.4, 19.5, 18.4, 16.7, 15.7, 10.6, 9.0. HRMS (EI) m/z Calcd, for C₅₁H₈₂AuNO₁₁P [M+H]⁺: 1112.5291, found: 1112.5290.

### DW-697

Following the general procedure using Macrolide Alkyne (100 mg), Chloro(triethylphosphine) gold (I) (39 mg) and potassium hydroxide (~8 mg) gave DW-697 (51 mg, 38% yield) as a white solid; ¹H NMR (400 MHZ, CDCl₃) δ 7.90 (d, J=8.1 Hz, 2H), 7.40 (d, J=8.2 Hz, 2H), 5.02 (d, J=4.6 Hz, 1H), 4.67 (d, J=10.3, 1H), 4.42 (d, 1H), 4.25 (d, J=6.1 Hz, 1H), 4.01 (m, 1H), 3.82 (d, 1H), 3.74-3.57 (m, 3H), 3.48-3.23 (m, 3H), 3.10 (s, 3H), 2.98 (d, J=7.8 Hz, 2H), 2.87 (m, 1H), 2.53 (m, 1H), 2.26 (s, 6H) 2.33 (d, J=14.9 Hz, 1H), 2.20 (S, 3H), 2.12-1.89 (m, 4H), 1.85-1.73 (s, 8H), 1.32-0.97 (m, 49H), 0.91-0.84 (m, 8H). HRMS (EI) m/z Calcd, for C55H98AuN3O12P [M+H]⁺: 1220.6554, found: 1220.6554.

### AW-07

Following the general procedure using CLM Alkyne (55 mg), Chloro(triethylphosphine) gold (I) (20 mg) and potassium hydroxide (4 mg) gave AW07 (20 mg, 27% yield) as a white solid; ¹H NMR (400 MHZ, CDCl₃) δ 7.42 (d, J=8.4 Hz, 2H), 7.13 (d, J=8.4 Hz, 2H), 5.4 (d, J=11.2 Hz, 1H), 4.90 (d, J=4.8 Hz, 1H), 4.40 (m, 1H), 3.95 (m, 2H), 3.77-3.65 (m, 3H), 3.61 (d, 1H), 3.44-3.20 (m, 2H), 3.18 (s, 1H), 3.12 (s, 3H), 3.01 (s, 5H), 2.98 (d, J=7.8 Hz, 2H), 2.87 (m, 1H), 2.53 (m, 1H), 2.33 (d, J=14.9 Hz, 1H), 2.20 (s, 3H), 1.89-1.69 (m, 12H), 1.38 (s, 3H), 1.37-0.92 (m, 35H) 0.83 (t, J=7.5 Hz, 3H). ¹³C NMR (176 MHZ, CDCl₃) δ 179, 138, 127, 124, 118, 109, 102, 94, 78, 77, 74, 73.7, 73, 70, 68, 65, 57, 53, 49.6, 49, 45, 42, 41, 37, 36, 34, 30, 29, 28, 26, 22, 21.71, 21, 18, 16, 14, 11, 9, 7. HRMS (EI) m/z Calcd, for C₅₂H₈₈AuNO₁₃P [M+H]⁺: 1162.5659, found: 1162.7856.

### Experimental Procedure and Characterization for Macrolide-Endoperoxide Conjugate

### Experimental Procedure & Characterization of DW-678

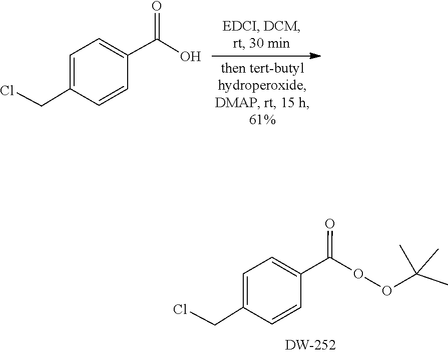 [SMILES: O=C(O)c1ccc(CCl)cc1 ; CC(C)(C)OOC(=O)c1ccc(CCl)cc1]

A mixture of 4-(chloromethyl)benzoic acid (1 g 1 equiv.) and EDCI.HCl (1.242 g, 1.1 equiv.) in dry CH₂Cl₂ (15 ml) was stirred at 0 °C in an argon atmosphere for 30 min. tert-butylhydroperoxide in 70 wt. % in H₂O (0.582 g, 2 equiv.) and DMAP (72 mg, 0.1 equiv.) were added and the reaction was stirred at rt for 15 h. TLC indicated the completion of the reaction. The reaction was quenched with water (50 mL), extracted with CH₂Cl₂ (2×30 mL), and the combined organic layer was sequentially washed with a saturated solution of NaHCO₃ and brine (30 mL). The organic layer was dried over Na₂SO₄, and then filtered. The solvent was removed using a rotary evaporator and the crude material was purified on silica gel column chromatography, eluting with Hexane:ethylacetate (9:1), v/v, to afford solid the desired product DW-252; yield: 870 mg (61%). ¹H NMR (700 MHZ, CDCl₃) δ 7.90 (d, J=8.5 Hz, 2H), 7.44 (d, J=6.3 Hz, 2H), 4.57 (s, 2H), 1.38 (s, 9H). ¹³C NMR (176 MHz, CDCl₃) δ 163.8, 142.8, 129.5, 128.7, 127.5, 84.0, 45.1, 26.2.

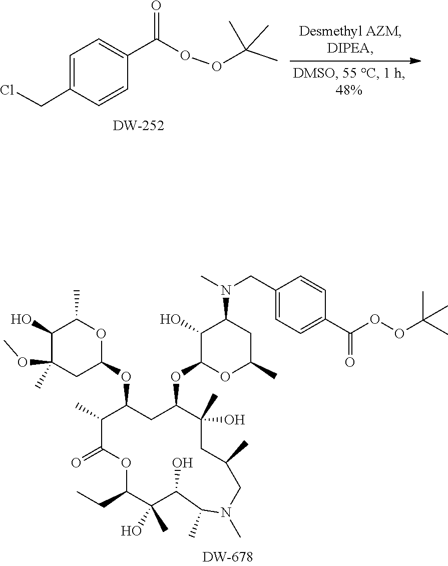 [SMILES: CC(C)(C)OOC(=O)c1ccc(CCl)cc1 ; CC[C@H]1OC(=O)[C@H](C)[C@@H](O[C@H]2C[C@@](C)(OC)[C@@H](O)[C@H](C)O2)C[C@@H](O[C@@H]2O[C@H](C)C[C@H](N(C)Cc3ccc(C(=O)OOC(C)(C)C)cc3)[C@H]2O)[C@](C)(O)C[C@@H](C)CN(C)[C@H](C)[C@@H](O)[C@]1(C)O]

A mixture of desmethylazithromycin (1.330 g, 1.1 equiv.) and DIPEA (0.830 mL, 3 equiv.) in dry DMSO (10 mL) was stirred for 10 min at rt. tert-butyl 4-(chloromethyl)benzoperoxoate (DW-252) (0.4 g, 1 equiv.) was added to the reaction, and the resulting mixture was stirred for 1 h at 55 °C. TLC indicated the completion of the reaction. The reaction was quenched with water (10 mL) and extracted with CH₂Cl₂ (3×50 mL) and the combined organic layer was sequentially washed with a saturated solution of NaHCO₃ and brine (30 mL). The organic layers dried over Na₂SO₄, and then filtered. The solvent was removed using a rotary evaporator, and the crude material was purified by silica gel column chromatography, eluting with CH₂Cl₂:Methanol (10:1), v/v, to furnish DW-678 as a white solid (yield=741 mg, 48%). ¹H NMR (400 MHZ, CDCl₃) δ 7.89 (dd, J=8.6, 2.1 Hz, 2H), 7.39 (d, J=8.4 Hz, 2H), 5.09 (s, 1H), 4.67 (d, J=9.7 Hz, 1H), 4.23 (s, 1H), 3.99 (m, 1H), 3.80 (d, 1H) 3.65 (m, 1H), 3.58 (d, J=13.6 Hz, 1H), 3.50 (d, J=13.6 Hz, 2H), 3.37-3.28 (m, 2H), 3.11 (s, 3H), 2.95 (m, 2H), 2.74-2.65 (m, 1H), 2.52 (d, J=11.8 Hz, 2H), 2.30 (s, 3H), 2.23 (s, 3H) 2.08-1.95 (m, 3H), 1.85-1.63 (m, 1H), 1.53-1.40 (s, 2H), 1.39 (S, 11H), 1.37-0.92 (m, 26H), 0.94-0.80 (s, 6H). ¹³C NMR (101 MHz, CDCl₃) δ 168.2, 163.8, 142.8, 129.5, 128.7, 127.5, 106, 103, 94, 84, 83, 78, 74, 73, 72, 70, 68, 65, 64, 62, 57, 54, 50, 49, 48, 45, 43, 42, 41 37, 36, 34, 29, 28, 27, 26, 22, 21, 18, 16, 14, 11, 9, 7. HRMS (EI) m/z Calcd, for C49H85N2O15 [M+H]⁺: 941.5950, found: 941.5930.

### Experimental Procedure & Characterization of DW-763

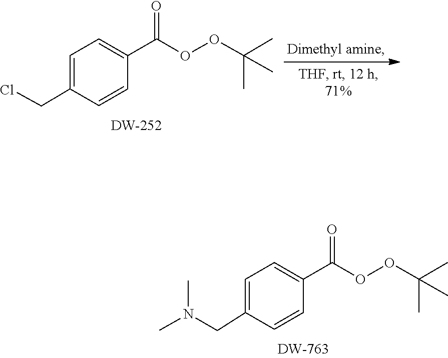 [SMILES: CC(C)(C)OOC(=O)c1ccc(CCl)cc1 ; CN(C)Cc1ccc(C(=O)OOC(C)(C)C)cc1]

A mixture of dimethyl amine (37 mg, 2 equiv.) in dry THF (1 mL) was stirred for 10 min at 0 °C tert-butyl 4-(chloromethyl)benzoperoxoate (DW-252) (100 mg, 1 equiv.) was dissolved in THF (1 mL), subsequently was added to the reaction and the resulting mixture was stirred at rt for 12 h. The completion of the reaction was indicated by TLC. The reaction mixture was quenched with water (10 mL) and extracted with CH₂Cl₂ (3×10 mL). The organic layers dried over Na₂SO₄, and then filtered. The solvent was removed using a rotary evaporator, and the crude material was purified by silica gel column chromatography, eluting with CH₂Cl₂:Methanol (10:1), v/v, to furnish DW-763 (yield=72 mg, 71%). ¹H NMR (400 MHZ, CDCl₃) δ 7.87 (d, J=4.5 Hz, 2H), 7.37 (d, J=8.0 Hz, 2H), 3.44 (s, 2H), 2.21 (s, 6H), 1.38 (s, 9H). 13C NMR (176 MHz, CDCl₃) δ 163.8, 142.8, 129.5, 128.7, 127.5, 84.0, 64.1, 45.1, 26.2.

### Experimental Procedure & Characterization of DW-854

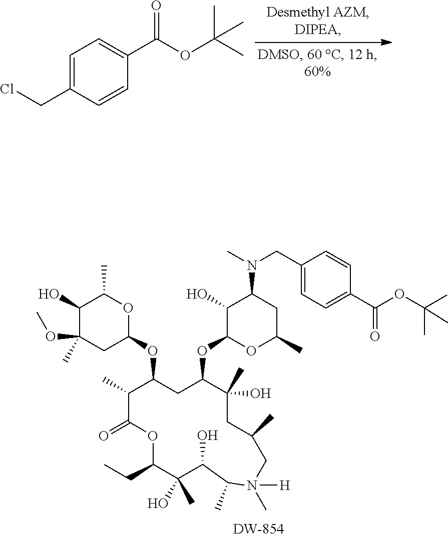

A mixture of desmethylazithromycin (0.2 g, 1 equiv.) and DIPEA (0.142 mL, 3 equiv.) in dry DMSO (2 mL) was stirred for 10 min at rt. tert-butyl 4-(chloromethyl)benzoate (0.61m g, 1 equiv.) was added to the reaction, and the resulting mixture was stirred for 12 h at 60 °C. TLC indicated the completion of the reaction. The reaction was quenched with water (10 mL) and extracted with CH₂Cl₂ (3×50 mL) and the combined organic layer was sequentially washed with a saturated solution of NaHCO₃ and brine (30 mL). The organic layers dried over Na₂SO₄, and then filtered. The solvent was removed using a rotary evaporator, and the crude material was purified by silica gel column chromatography, eluting with CH₂Cl₂:Methanol (10:1), v/v, to furnish DW-854 as a white solid (yield=152 mg, 60%). ¹H NMR (400 MHZ, CDCl₃) δ 7.94 (d, J=8.2 Hz, 2H), 7.35 (d, J=8.3 Hz, 2H), 5.04 (s, 1H), 4.65 (d, J=9.7 Hz, 1H), 4.41 (m, 1H), 4.24 (s, 1H), 4.0 (m, 1H), 3.80 (m, 2H), 3.63 (m, 1H), 3.53-3.42 (s, 2H), 3.40-3.30 (d, J=4.8 Hz, 2H), 3.14-3.07 (s, 3H), 2.83-2.75 (s, 2H), 2.56-2.45 (s, 1H), 2.33-2.26 (s, 3H), 2.26-2.20 (s, 3H), 2.09-1.69 (m, 5H), 1.61-1.54 (s, 11H), 1.37-0.92 (m, 26H), 0.91-0.87 (m, 6H). ¹³C NMR (101 MHZ, CDCl₃) δ 167.2, 163.4, 142.3, 129.3, 128.4, 127.2, 106, 103, 94, 84, 83, 78, 74, 73, 72, 70, 68, 65, 64, 62, 57, 54, 50, 49, 48, 45, 43, 42, 41 37, 36, 34, 29, 28, 27, 26, 22, 21, 18, 16, 14, 11, 9, 7. HRMS (EI) m/z Calcd, for C49H85N2O14 [M+H]⁺: 925.6001, found: 925.6012.

### Experimental Procedure & Characterization of DW-857

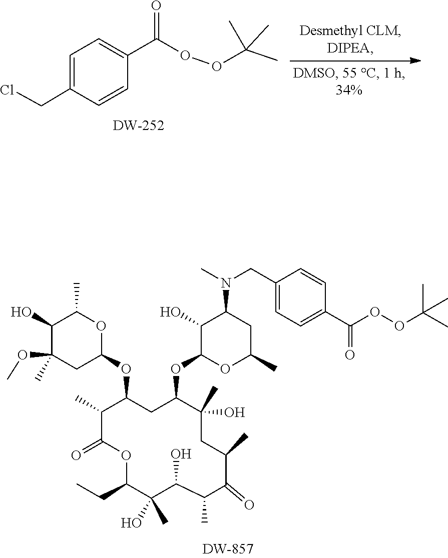

A mixture of desmethylclarithromycin (0.2 g, 1 equiv.) and DIPEA (0.138 mL, 3 equiv.) in dry DMSO (2 mL) was stirred for 10 min at rt. tert-butyl 4-(chloromethyl)benzoperoxoate (DW-252) (66 mg, 1 equiv.) was added to the reaction, and the resulting mixture was stirred for 1 h at 55 °C. TLC indicated the completion of the reaction. The reaction was quenched with water (20 mL) and extracted with CH₂Cl₂ (3×20 mL) and the combined organic layer was sequentially washed with a saturated solution of NaHCO₃ and brine (30 mL). The organic layers dried over Na₂SO₄, and then filtered. The solvent was removed using a rotary evaporator, and the crude material was purified by silica gel column chromatography, eluting with CH₂Cl₂:Methanol (10:1), v/v, to furnish DW-857 as a white solid (yield=87 mg, 34%). Partial NMR: ¹H NMR (400 MHZ, CDCl₃) δ 8.10 (dd, J=8.6, 2.1 Hz, 2H), 7.51 (d, J=8.4 Hz, 2H), 5.09 (s, 1H), 4.67 (d, J=9.7 Hz, 1H), 4.23 (s, 1H), 3.99 (m, 1H), 3.80 (d, 1H) 3.65 (m, 1H), 3.58 (d, J=13.6 Hz, 1H), 3.50 (d, J=13.6 Hz, 2H), 3.37-3.28 (m, 2H), 3.11 (s, 3H), 2.95 (m, 2H), 2.74-2.65 (m, 1H), 2.52 (d, J=11.8 Hz, 2H), 2.30 (s, 3H), 2.23 (s, 3H) 2.08-1.95 (m, 3H), 1.85-1.63 (m, 1H), 1.53-1.40 (s, 2H), 1.39 (S, 11H), 1.34-1.00 (m, 26H), 0.87-0.67 (d, J=8.3 Hz, 3H).

### Example 3—Biological Data for Macrocycle ROS Inducers

### Cell Culture and Cell Viability Assays

The human breast (MCF-7, MDA-MB-231, and MDA-MB-453), prostate (C4-2B and 22Rv1), and lung (A549) cancer cell lines, along with normal cell lines (Vero) were obtained from American Type Culture Collection (ATCC). Cell lines were verified by ATCC and only used while passage numbers were low (<20). MDA-MB-453, C4-2B, and 22Rv1 cell lines were routinely cultured in phenol-free RPMI (Invitrogen) with fetal bovine serum (10%), L-Glutamine (1%) and penicillin-streptomycin (1%); MCF-7 cell line was maintained in phenol red-free DMEM (Invitrogen) with fetal bovine serum (10%), L-Glutamine (1%) and penicillin-streptomycin (1%); Vero and MDA-MB-231 cell lines were maintained in phenol red-containing DMEM (Invitrogen) with fetal bovine serum (10%) and penicillin-streptomycin (1%); and A459 cell line was maintained in phenol red-containing DMEM (Invitrogen) with fetal bovine serum (10%), L-Glutamine (1%) and penicillin-streptomycin (1%). All cell cultures were incubated at 37 °C under a humidified atmosphere of 5% CO₂ and 95% air. For all experiments, cells were grown in 6 and/or 96-well culture treated microtiter plates (Techno Plastic Product AG) with the appropriate ligand in triplicates for 72 h. MTT (475989-10GM, EMD Millipore Corp., USA) was employed to determine cell viability per the manufacturers' instructions. Briefly, cells were seeded in 96-well flat-bottom microtiter plates (transparent) at a density of 4500 cells per well (100 μL) and incubated for 24 h to allow the cells to adhere and resume exponential growth. After this, the wells were drained and then treated with eight concentrations of lead compounds (0.5, 1.25, 2.5, 5.0, 12.5, 25, 50, 100 μM) and incubated for 72 h. After the incubation time, 10 μL of MTT reagent was added to each well of the microtiter plates containing treated cells, untreated cells (negative control) and cell culture medium—the blank (background control) and incubated for 3 h. The growth media-MTT reagent suspension was aspirated after 3 h incubation period, and replaced with 100 μL DMSO, and then incubated for 15 min to completely dissolve formazan (insoluble crystal) resulting from the reductive action of NADPH-dependent dehydrogenase on tetrazolium salt of MTT. After this, the absorbance was measured using the iTecan plate reader at 570 nm. The effects of treatment on cell proliferation and survival were expressed as:

$$Percentage cell viability = \frac{OD of treatment - OD of background control}{OD of negative control - OD of background control} \times 100$$

(where OD=optical density). IC₅₀ values were calculated by non-linear regression (log [concentration of inhibitor]versus response (% cell viability) using the GraphPad Prism analysis software. For the calculation of Mean IC₅₀ values, triplicate runs (n=3) were selected.

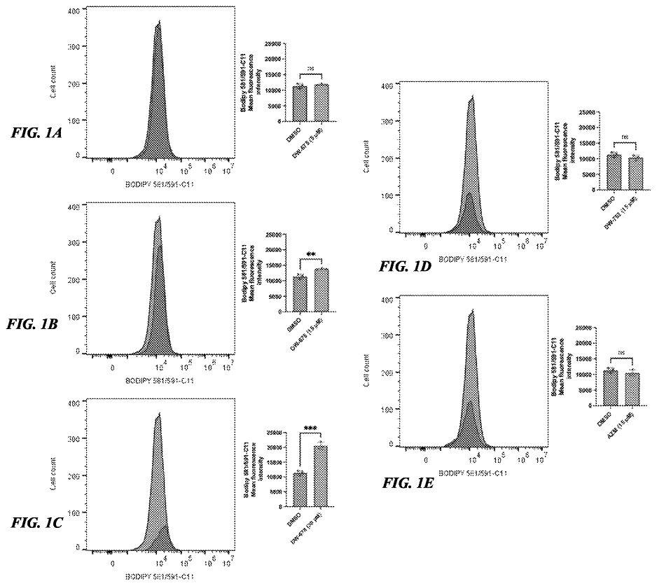

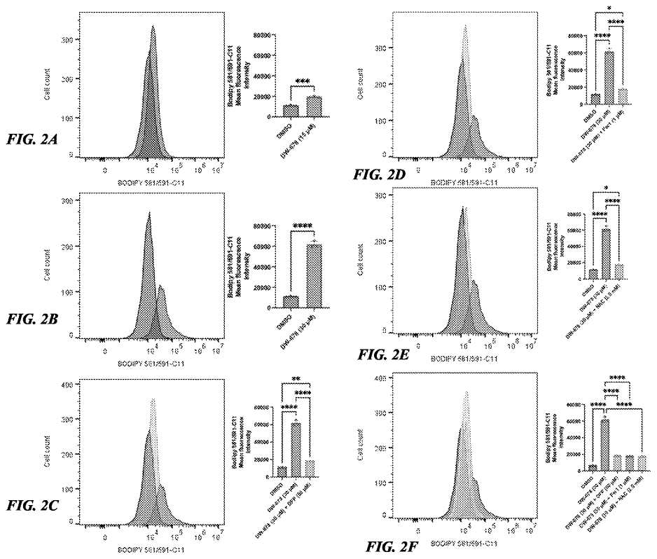

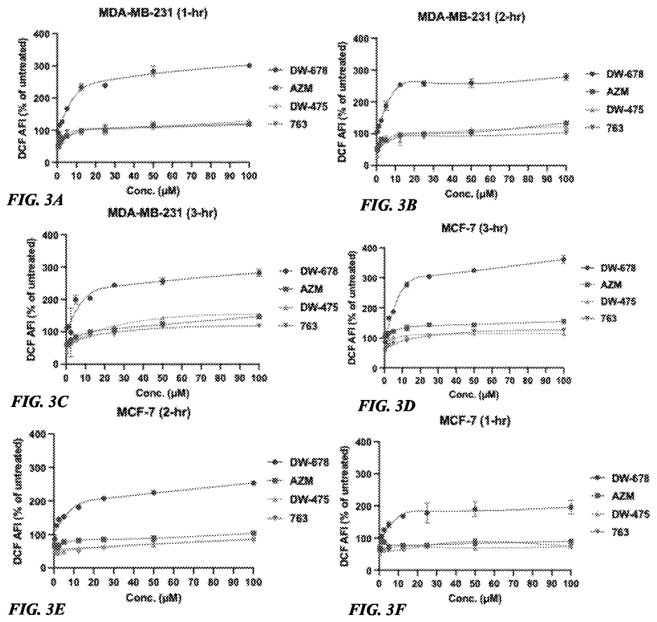

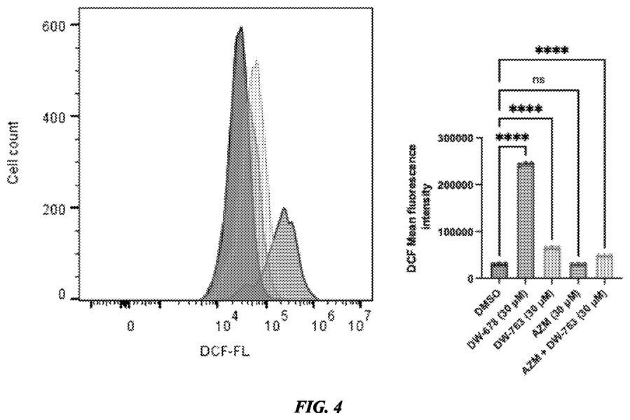

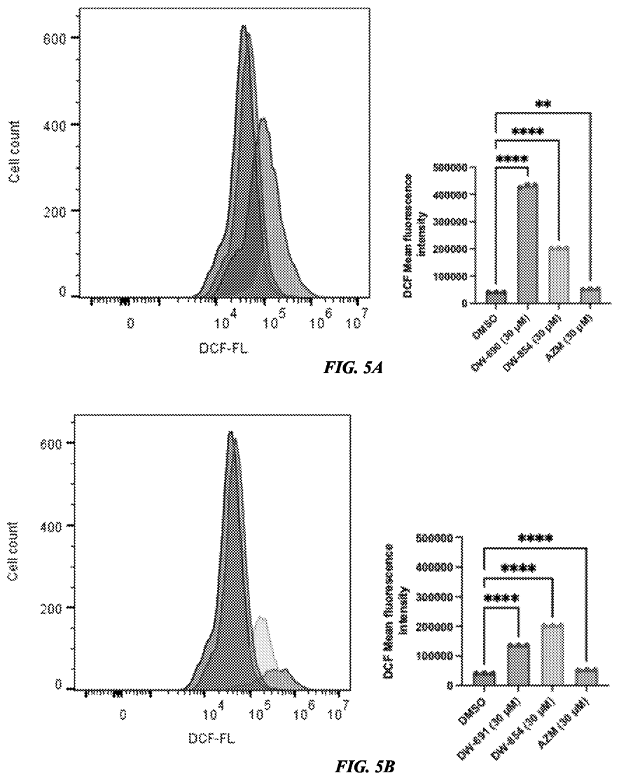

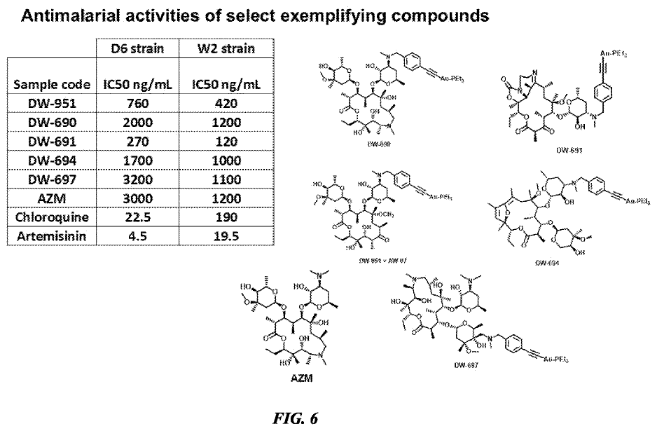

As shown in Table 1, the exemplifying compounds inhibit the proliferation of cancer cells. Several of these compounds are more toxic to the tested cancer cell lines relative to the normal Vero cells. Ferroptosis induction was confirmed using representative compound DW-678 (FIGS. 1A-1E and FIGS. 2A-2F) while ROS induction was confirmed using representative compound DW-678 (FIGS. 3A-3F and FIG. 4), and compounds DW-690, DW-691 and DW-854 (FIGS. 5A-5B). Moreover, a representative cohort among these compounds also inhibit the viability of both the wildtype and drug-resistant strains of P. falciparum (FIG. 6, Table 2).

TABLE 1
Effect of exemplifying Formula I compounds on cell proliferation. Note that DW-
763, Azithromycin (AZM) and Clarithromycin (CLM) were used as control compounds

| | IC₅₀ (μM)-72 hours | | | | | | |
|---|---|---|---|---|---|---|---|
| MTT | C4-2B | 22Rv1 | MCF-7 | MDA-MB-231 | MDA-MB-453 | A549 | Vero |
| AW-07 | 1.01 ± 0.07 | 0.90 ± 0.45 | 2.32 ± 0.45 | 0.65 ± 0.14 | 0.67 ± 0.04 | 14.98 ± 2.17 | 3.44 ± 0.22 |
| DW-690 | 1.31 ± 0.15 | 1.83 ± 1.00 | 3.40 ± 0.14 | 2.20 ± 0.72 | 1.39 ± 0.13 | 12.48 ± 0.16 | 7.11 ± 0.68 |
| DW-691 | 0.78 ± 0.21 | 0.95 ± 0.54 | 2.40 ± 0.54 | 0.66 ± 0.07 | 0.69 ± 0.04 | 10.33 ± 1.63 | 2.66 ± 0.03 |
| DW-694 | 7.34 ± 0.91 | 8.90 ± 0.41 | 28.82 ± 0.57 | 7.56 ± 1.17 | 6.64 ± 0.41 | >100 | 29.73 ± 0.59 |
| DW-697 | 0.61 ± 0.02 | 0.60 ± 0.09 | 2.04 ± 0.58 | 1.23 ± 0.24 | 0.70 ± 0.08 | 7.49 ± 0.04 | 1.61 ± 0.13 |
| DW-678 | NYT | NYT | 3.28 ± 0.62 | 4.81 ± 0.35 | 7.29 ± 0.61 | NYT | 3.56 ± 0.44 |
| DW-854 | 9.83 ± 0.21 | 6.49 ± 0.28 | 3.96 ± 0.79 | 7.18 ± 0.32 | 8.43 ± 0.32 | 6.02 ± 0.35 | 8.19 ± 1.28 |
| DW-763 | NYT | NYT | 23.18 ± 2.01 | 41.38 ± 3.77 | 30.84 ± 4.91 | NYT | >100 |
| AZM | >100 | >100 | >100 | >100 | >100 | >100 | >100 |
| CLM | >100 | >100 | >100 | >100 | >100 | >100 | >100 |

NYT = Not Yet Tested

TABLE 2
Antimalarial Activities of Select Exemplifying Compounds

| | D6 strain | W2 strain |
|---|---|---|
| Sample Code | IC₅₀ ng/mL | IC₅₀ ng/mL |
| DW-951 (AW-07) | 760 | 420 |
| DW-690 | 2000 | 1200 |
| DW-691 | 270 | 120 |
| DW-694 | 1700 | 1000 |
| DW697 | 3200 | 1100 |
| AZM | 3000 | 1200 |
| Chloroquine | 22.5 | 190 |
| Artemisinin | 4.5 | 19.5 |

What is claimed is:

1. A compound of formula (I) having the following structure: 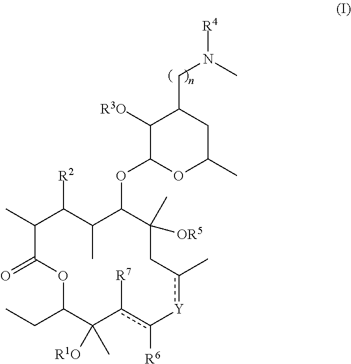
or a stereoisomer, pharmaceutically acceptable salt, oxide, or solvate thereof, wherein
R¹, is a bond, H, or C₁-C₆ alkyl;
R² is oxo or —OR⁸;
R³, R⁵, and R⁶ are each independently H or C₁-C₆ alkyl;
R⁴ is H or C₁-C₆ alkyl, wherein C₁-C₆ alkyl can be substituted with 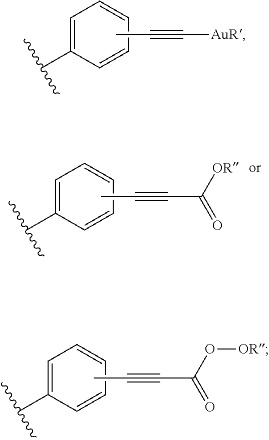
R⁷ is H, hydroxyl, C₁-C₆ alkyl, C₁-C₆ alkoxy, —NR^{a}R^{b},
R⁸ is H, C₁-C₆ alkyl, or 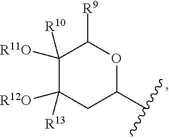
R⁹, R¹¹, R¹², and R¹³ are each independently H or C₁-C₆ alkyl;
R¹⁰ is H, C₁-C₆ alkyl, or 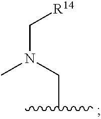
R¹⁴ is H or C₁-C₆ alkyl, wherein C₁-C₆ alkyl can be substituted with 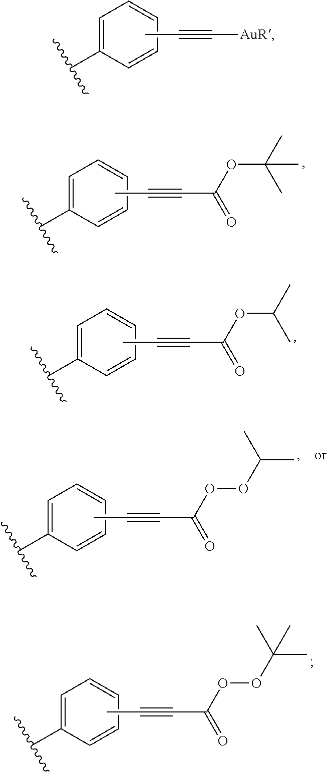
Y is —NR¹⁵CH₂, C═O, C, C═NR^{c}, or CH₂;
n is 0 or 1; [symbol]
is a single or double bond;
R¹⁵ is H or C₁-C₆ alkyl; and
R′ is 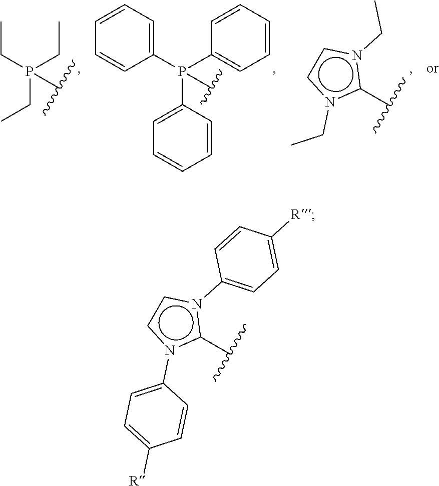
R″ is branched or unbranched C₁₋₆ alkyl;
R′″ is H, C₁₋₆ alkyl, halogen, or —OCH₃
R^{a} H or C₁₋₆ alkyl, wherein C₁₋₆ alkyl can be substituted with oxo;
R^{b} is H or C₁₋₆ alkyl;
R^{c} is H or C₁₋₆ alkyl; or
R¹ and R^{a}, and R^{b} and Re come together with the atoms to which they are attached to form a tricyclic ring with the macrocycle of formula (Ia); or
Y is C and R¹ is a bond connected to Y to form a bicyclic ring with the macrocycle of formula (Ia).

2. A compound of formula (I) having the following structure: 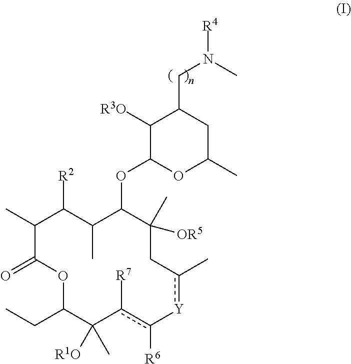
or a stereoisomer, pharmaceutically acceptable salt, oxide, or solvate thereof, wherein
R¹, is a bond, H, or C₁-C₆ alkyl;
R² is oxo or —OR⁸;
R³, R⁵, and R⁶ are each independently H or C₁-C₆ alkyl;
R⁴ is H or C₁-C₆ alkyl, wherein C₁-C₆ alkyl can be substituted with 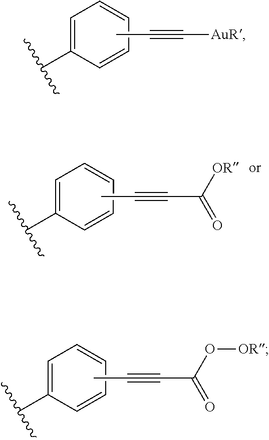
R⁷ is H, hydroxyl, C₁-C₆ alkyl, C₁-C₆ alkoxy, —NR^{a}R^{b};
R⁸ is H, C₁-C₆ alkyl, or 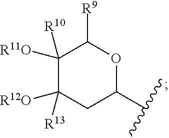
R⁹, R¹¹, R¹², and R¹³ are each independently H or C₁-C₆ alkyl;
R¹⁰ is H, C₁-C₆ alkyl, or 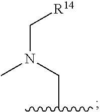
R¹⁴ is H or C₁-C₆ alkyl, wherein C₁-C₆ alkyl can be substituted with 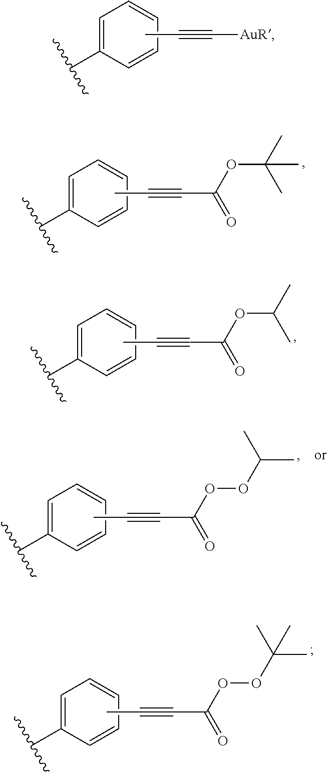
Y is —NR¹⁵CH₂—, C═O, C═NR^{c}, or CH₂;
n is 0 or 1; [symbol]
is a single or double bond;
R¹⁵ is H or C₁-C₆ alkyl; and
R′ is 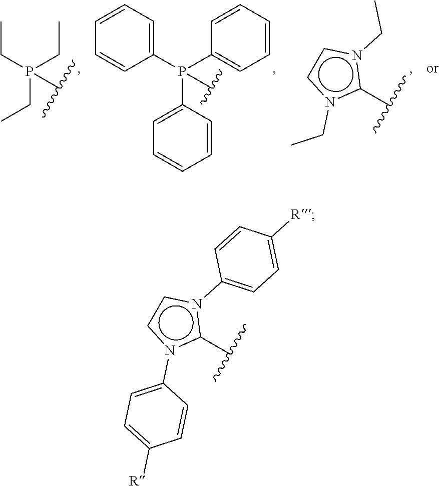
R″ is branched or unbranched C₁₋₆ alkyl;
R′″ is H, C₁₋₆ alkyl, halogen, or —OCH₃
R^{a} H or C₁₋₆ alkyl, wherein C₁₋₆ alkyl can be substituted with oxo;
R^{b} is H or C₁₋₆ alkyl;
R^{c} is H or C₁₋₆ alkyl; or
R¹ and R^{a}, and R^{b} and Re come together with the atoms to which they are attached to form a tricyclic ring with the macrocycle of formula (I); or
R¹ is a bond connected to Y to form a bicyclic ring with the macrocycle of formula (I).

3. The compound according to claim 1, wherein
R⁴ is C₁-C₆ alkyl substituted with

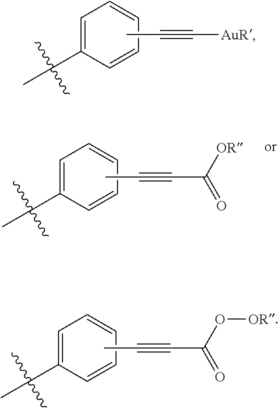

4. The compound according to claim 3, wherein n is 0.

5. The compound according to claim 3, wherein
R² is-OR⁸;
R⁸ is 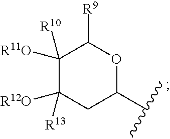
and
R¹⁰ is H.

6. The compound according to claim 3, wherein
R² is oxo.

7. The compound according to claim 1, wherein n is 1.

8. The compound according to claim 7, wherein
R² is —OR⁸;
R⁸ is 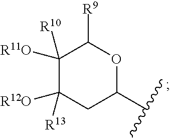
R¹⁰ is 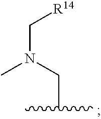
and
R¹⁴ is C₁-C₆ alkyl substituted with

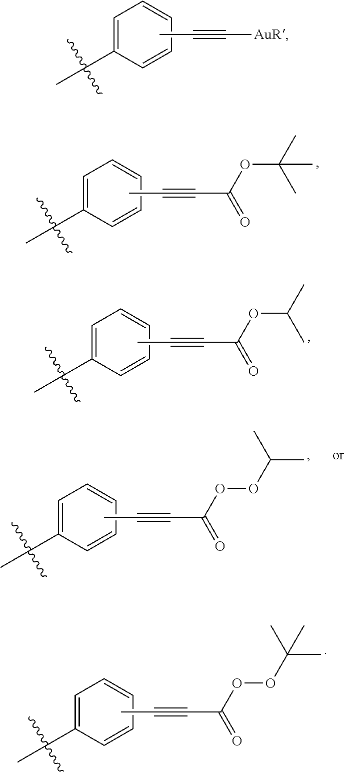

9. The compound according to claim 1, selected from the group consisting of

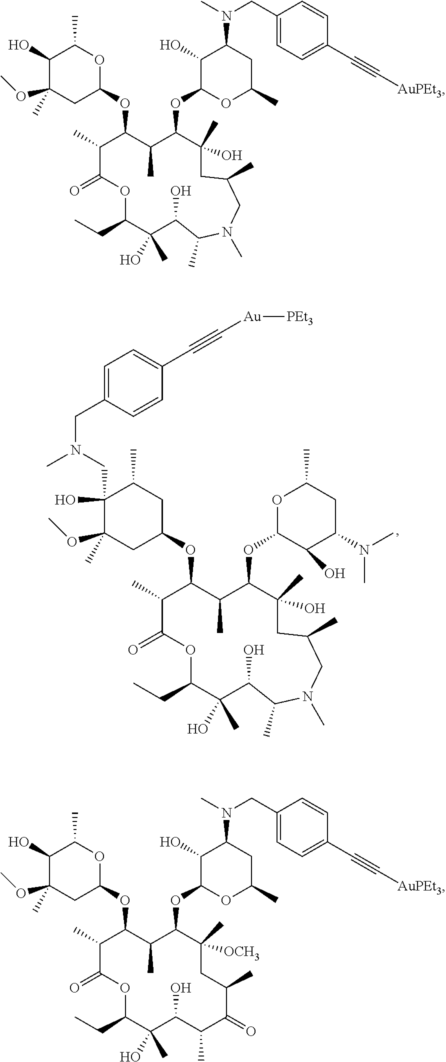

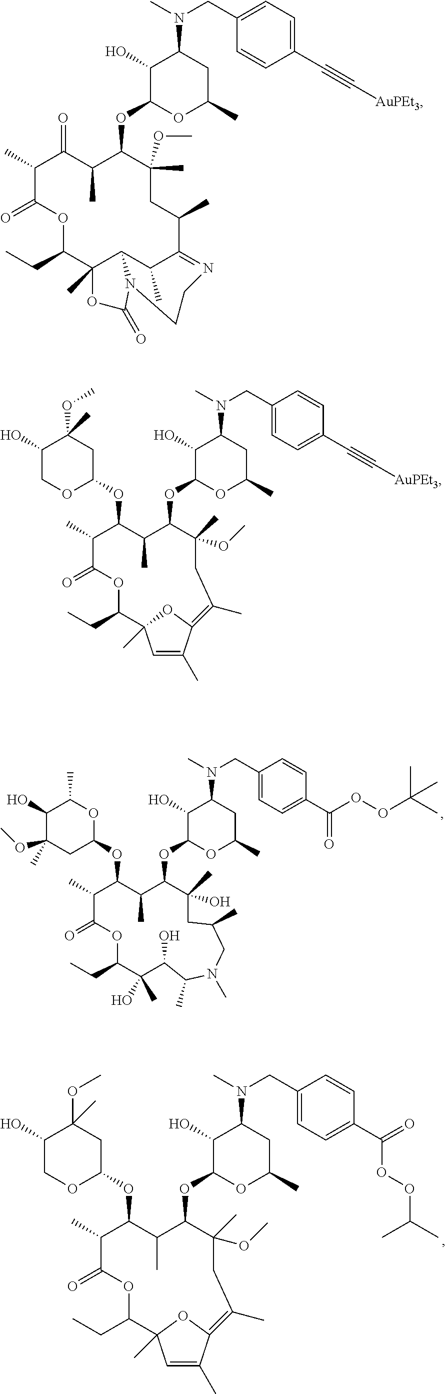

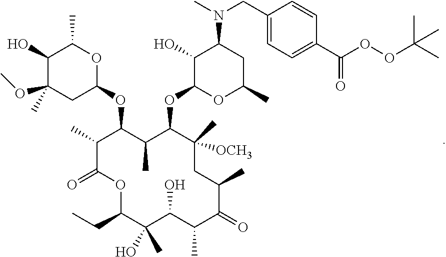 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)Cc3ccc(C(=O)OOC(C)(C)C)cc3)[C@H]2O)[C@](C)(OC)C[C@@H](C)C(=O)[C@H](C)[C@@H](O)[C@]1(C)O]

10. A method of treating a cancer in a subject, said method comprising:
providing a compound according to claim 1, to a subject in need of said treatment.

11. The method of claim 10, wherein the cancer is selected from the group consisting of prostate cancer, lung cancer, liver cancer, or breast cancer.

12. A method of up-regulating reactive oxygen species (ROS) in a cancer cell, said method comprising:
contacting the cancer cell with a compound according to claim 1, under conditions effective to up-regulate reactive oxygen species in the cell.

13. The method of claim 12, wherein the cancer cell is selected from the group consisting of a prostate, lung, liver, or breast cancer cell.

14. The method of claim 12, wherein up-regulating reactive oxygen species in the cancer cell promotes ferroptosis of the cancer cell.

15. The method of claim 12, wherein up-regulating reactive oxygen species in the cancer cell inhibits proliferation of the cancer cell.

16. A method of treating malaria in a subject, said method comprising:
providing a compound according to claim 1, to a subject in need of said treatment.

17. A composition comprising:
a compound according to claim 1; and
a carrier.
